(19) United States
(12) Patent Application Publication
(10) Pub. No.: US 2014/0200567 A1
(43) Pub. Date: Jul. 17, 2014

(54) METHODS AND DEVICES FOR THE TREATMENT OF ATRIAL FIBRILLATION

(71) Applicant: Adagio Medical, Inc., Laguna Hills, CA (US)

(72) Inventors: James L. Cox, Denver, CO (US); Jay J. Eum, Irvine, CA (US)

(73) Assignee: Adagio Medical, Inc., Laguna Hills, CA (US)

(21) Appl. No.: 14/158,549

(22) Filed: Jan. 17, 2014

Related U.S. Application Data
(63) Continuation of application No. PCT/US2012/047487, filed on Jul. 19, 2012.
(60) Provisional application No. 61/572,611, filed on Jul. 19, 2011.

(51) Int. Cl. A61B 18/02

(52) U.S. Cl. CPC A61B 18/02; A61B 18/0206
USPC 606/21

(57) ABSTRACT

Apparatus, systems and methods for creation of ablation lesions for the treatment of atrial fibrillation. A method for creating a maze of lesions to isolate macro re-entrant circuits. An ablation catheter with at least one ablation surface at its distal end. A flexible ablation probe with at least one ablation surface at its distal end. A clamp with opposing jaws having at least one jaw with an ablation surface, optionally including temperature sensing.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### FIELD

The present disclosure relates generally to medical devices, systems and methods for treating atrial fibrillation by creating transmural lesions in the heart.

### BACKGROUND

### Introduction

Atrial fibrillation (“AF”) is the most common cardiac arrhythmia causing the muscles of the atria to contract in an irregular quivering motion rather than in the coordinated contraction that occurs during normal cardiac rhythm. Multiple studies have been performed over the past 30 years to determine the incidence of AF in the general population with results varying between 0.4% and 2.0% and it is generally accepted by most authorities that approximately 1% of the general population of any country/region has AF. This means that approximately 3 million people in the USA have AF with another 3 million or so in Western Europe. Since the world's population is approximately 6.5 billion, it would be expected that at least 60 million people in the world have AF, each country's incidence being closely related to that country's life expectancy.

AF may be detected by the presence of an irregular pulse or by the absence of p-waves on an electrocardiogram. During an episode of AF, the regular electrical impulses that are normally generated by the sinoatrial (SA) node are overwhelmed by rapid disorganized electrical impulses in the atria. These disorganized impulses are induced by “triggers” that are usually, though not always, located in and around the orifices of the pulmonary veins. Because the resultant disorganized impulses of AF reach the atrioventricular (AV) node in a rapid (up to 600 per minute) and highly irregular manner, the impulses that are subsequently filtered and conducted through the AV node to the ventricles are also rapid (around 150 per minute) and said to be “irregularly irregular.”

Although patients do not usually experience immediate life-threatening problems from the onset of AF, they commonly experience immediate symptoms such as palpitations (irregularity) of the heart, weakness, tiredness and shortness of breath. In patients with other concomitant heart disease, congestive heart failure may result when AF occurs. The most serious complication of AF is the risk of stroke caused by the pooling and stasis of blood in the left atrial appendage (LAA) that results in the formation of clots that may break off and travel to the brain. AF is second only to arteriosclerosis as a cause of strokes and is responsible for hundreds of thousands of strokes in the US alone each year.

AF may be treated with medications which either slow the heart rate or convert the heart rhythm back to normal. Synchronized electrical cardioversion may also be used to convert AF to a normal heart rhythm but the simple conversion does not actually address the underlying cause of the AF and, therefore, is usually only a temporary stop-gap measure. Surgical and catheter-based therapies (“interventional therapies”) may also be used to treat AF in certain individuals. Over one million patients have had catheter and/or surgical interventional therapy; however, this represents less than 2% of the total population of AF patients in the Western world. Catheter ablation has attained long-term success in only 29% of patients after one catheter ablation and in only about 60% of patients after multiple ablations. Surgical intervention for AF is somewhat more successful but in general is too invasive to be widely applied.

### Classification and Treatment of Atrial Fibrillation

AF episodes may be intermittent (“paroxysmal”) lasting from minutes to weeks or they may last for years, in which case the AF may be classified as continuous or “persistent.” Recently, the American Heart Association (“AHA”), American College of Cardiology (“ACC”) and the European Cardiology Society (“ECS”) adopted a new classification system for AF, which includes Paroxysmal AF (“PAF”), Persistent AF, Long-Standing (“L-S”) Persistent AF and Permanent AF. The latter three types of AF are sometimes referred to as “chronic AF” or “Non-Paroxysmal AF” (Non-PAF). 60% of all AF is paroxysmal and 40% is non-paroxysmal. The underlying electrophysiology differs between paroxysmal (intermittent) AF and chronic AF as does the interventional treatment strategies.

Patients who have Paroxysmal Atrial Fibrillation (“PAF”) usually spend most of their time in normal sinus rhythm (“NSR”). They then experience a premature atrial beat (“trigger”) that induces atrial macro-reentry, which is the electrical state of the atrium during the actual episode of AF. These self-perpetuating macro-reentrant circuits continue until they either stop spontaneously or are terminated by drugs. The patient then resumes NSR until another episode of AF is induced by a trigger. Thus, the nature of the PAF cycle may be described as being induced by the atrial triggers and maintained by the macro-reentrant circuits. Because of a phenomenon called “atrial remodeling,” the self-perpetuating macro-reentrant circuits can become so stable that they do not spontaneously terminate, thereby causing AF to persist. According to Haissaguerre (New England Journal of Medicine 1998; 339:659), the full contents of which are incorporated herein by reference, in 90% of cases the triggers are located in and around the pulmonary vein orifices in the left atrium, while the other 10% of triggers are located in areas of the atrium remote from the pulmonary veins.

Persistent, L-S Persistent and Permanent AF (all “Non-paroxysmal” types of AF) are sustained for longer periods of time by macro-reentrant “drivers” that become self-perpetuating with time, probably due to atrial remodeling. Since all three forms of these non-paroxysmal types of AF depend upon a different mechanism (macro-reentry) than that of paroxysmal AF (focal triggers), interventional treatment in these three groups of patients generally involves both isolating triggers and macro-reentry circuit disruption.

For practical purposes, when classifying patients who undergo interventional therapy, such as catheter ablation or surgery, all AF may be divided into PAF and Non-PAF because the underlying mechanisms and the interventional treatment are specific to those two groups of patients. Interventional treatment of PAF involves PV isolation, while interventional treatment of Non-PAF involves PV isolation as well as additional linear lesions.

TABLE 1
Classification of AF.

| AHA/ACC/ECS | UNDERLYING | INTERVENTIONAL | INTERVENTIONAL |
|---|---|---|---|
| CLASSIFICATION | ELECTROPHYSIOLOGY | TREATMENT | CLASSIFICATION |
| Paroxysmal | Focal “Triggers” | PV Isolation | PAF |
| Persistent | Macro-Reentrant | Additional Linear | Non-PAF |
| L-S Persistent | “Drivers” | Lesions to Ablate | |
| Permanent | | Macro-Reentrant | |
| | | “Drivers” | |

In addition to being classified as either PAF or Non-PAF, AF patients fall into four possible pre-operative categories. If the AF is associated with cardiac disease that in and of itself warrants surgery, the AF is said to be “concomitant”. Thus, patients who are to undergo mitral valve surgery, aortic valve surgery, coronary bypass surgery or left heart failure surgery who also have AF are said to have “Concomitant AF”. If their AF is paroxysmal, they fall into the category of “Concomitant PAF”. If their AF is non-paroxysmal, they fall into the category of “Concomitant Non-PAF”. Patients who have AF but do not have associated heart disease that is severe enough to warrant surgery are said to have “Stand-Alone AF”. If their AF is paroxysmal, they fall into the category of “Stand-Alone PAF”. If their AF is non-paroxysmal, they fall into the category of “Stand-Alone Non-PAF”.

There are currently about 60,000 patients entering operating rooms in the USA each year with Concomitant AF-AF associated with other cardiac disease warranting surgery (about 2% of the total AF population). Approximately 30,000 of them receive concomitant AF surgical procedures (PV Isolation, the Maze Procedure or some modification thereof) annually. There are approximately three million Stand-alone AF patients who have no other cardiac disease severe enough to warrant surgery. These patients represent potential market for interventional AF treatment by catheter ablation because surgical intervention remains generally too invasive for Stand-Alone AF. However, only the simpler types of Stand-Alone PAF patients have thus far been treated successfully by catheter ablation, about 5% penetrance of the market, with only a 1% penetrance of the more difficult to treat Stand-Alone Non-PAF market. The overall penetrance of interventional electrophysiology in the Stand-Alone AF market is currently estimated to be about 3% and their success rate after one catheter ablation is approximately 29%. Success can be increased to about 60% with two or more individual sessions of catheter ablation. On the other hand, interventional surgery for stand-alone AF is extremely rare but also highly successful, with reports of over 90% success in several different series.

Several studies have evaluated the efficacy of different interventional techniques for AF. The traditional Cox Maze procedure involves cutting the atrial wall with a scalpel in particular patterns that isolate the foci of arrhythmia and then sewing the cardiac tissue back together. Upon healing, the resultant scar tissue serves to interrupt ectopic re-entry pathways and other aberrant electrical conduction thus preventing arrhythmia and fibrillation. In 2003, Damiano, et al. (Journal of Thoracic Cardiovascular Surgery 2003; 126(6):2016-21), the full contents of which are incorporated herein by reference, reported the results of the surgical cut-and-sew Maze procedures performed in the 1990's. After fifteen years 92% of the patients who had undergone Stand-Alone Maze procedures were still free of AF. Of the patients who had undergone Concomitant Maze procedures, 97% were still free of AF after ten years. These results are commonly referred to as the “gold standard” for the interventional treatment of AF. While the surgical Cox Maze procedure has a high success rate, it is a difficult to perform open chest/open atrium procedure requiring the heart to be stopped and the establishment of a coronary bypass. These limitations cannot be overcome by interventional electrophysiology techniques as the full bi-atrial Maze procedure cannot be performed using current catheter ablation techniques. Even when minimally invasive surgical techniques are employed, the full bi-atrial Maze procedure requires use of a heart-lung machine. It is therefore reserved for severe cases of AF or cases where the AF is associated with cardiac disease that in and of itself warrants surgery, “concomitant” AF.

Most surgeons and all interventional electrophysiologists using minimally invasive surgical and catheter ablation techniques perform some procedure less than the full bi-lateral Maze procedure. Predominantly, “left-atrial only” procedures, such as Pulmonary Vein (PV) Isolation, a so-called “left-sided Maze” procedure, a so-called “modified Maze” procedure, and any number of different “Hybrid” procedures, which employ both surgical and electrophysiology techniques, are performed. Catheter ablation is successful for highly-selected patients with simple forms of PAF, but overall, interventional therapies employing catheter ablation are about 60% successful after multiple ablation sessions. The same low overall success rate is obtained for left-sided surgical procedures. In 2006, Dong, et al. (Journal of Cardiovascular Electrophysiology, 17: 1080-10850 the full contents of which are incorporated herein by reference, reported a 28% two-year success rate for a one-time catheter ablation procedure in 200 patients, where patients were roughly half PAF and half Non-PAF. In January 2011, Weerasooriya, et al. (Journal of the American College of Cardiology, 2011; 57:160-166), the full contents of which are incorporated herein by reference, reported a success rate of 29% at five years after a single ablation and 63% after two or more separate catheter ablations with 75% of the patient population having PAF. These kinds of results for the catheter ablation of AF in highly selected patients after over 15 years of experience in over one million patients indicate that better approaches to interventional AF therapy are needed.

In summary, drug therapy is notoriously suboptimal for AF and there is no satisfactory interventional therapy for more than 97% of patients with AF. Catheter ablation has poor results in these patients and even so-called “minimally invasive” surgery is too invasive to be used routinely.

### SUMMARY

Several embodiments described herein relate to systems, methods, and medical devices for providing minimally invasive interventional treatment of all forms of AF with a pattern of conduction-blocking lesions in the heart comprising a first conduction-blocking lesion extending along a line between the inferior and superior vena cava, a second conduction-blocking lesion extending transversely across the right atrium and intersecting the first conduction-blocking lesion between the inferior and superior vena cava, a third conduction-blocking lesion extending laterally along the right atrium and intersecting the second conduction-blocking lesion, a fourth conduction-blocking lesion in the coronary sinus, a fifth conduction-blocking lesion extending along a transverse line located below the right and left inferior pulmonary veins, a sixth conduction-blocking lesion extending along a transverse line located above the right and left superior pulmonary veins, a seventh conduction-blocking lesion comprised of a plurality of lesions extending along the anterior interatrial groove proximate the origins of the right superior and inferior pulmonary veins and intersecting the fifth conduction-blocking lesion below the right inferior pulmonary vein and the sixth conduction-blocking lesion above the right superior pulmonary vein and an eighth conduction-blocking lesion located along a line extending from the base of the left atrial appendage to a location proximate the mitral annulus. Lesions may be made in any order. In some embodiments, one or more lesions may be made concomitantly.

In some embodiments, an off-pump, minimally invasive maze procedure is performed using a variety of tools and techniques resulting in transmural lesions sufficient to cure AF. Procedures may be performed using one or more standard access techniques such as endoscopy, catheter access, and small surgical incisions (“mini-thorocotomies”). In some embodiments, a combination of catheter access, endoscopy and thorocotomy is used to minimally invasively access the right and left sides of the heart to produce the desired lesions. In some embodiments, right side access may be used to produce lesions between the superior and inferior vena cava, along the right atrium, and along the coronary sinus. In some embodiments, left side access is used to produce lesions between the superior and inferior left pulmonary veins, for isolation of the right pulmonary vein, and for lesions along the left atrial appendage.

In some embodiments, a minimally invasive method of providing interventional treatment of AF with a pattern of conduction-blocking lesions comprising making in any order a series of lesions comprising making a first lesion extending along a line between the inferior and superior vena cava, making a second lesion extending transversely across the right atrium and intersecting the first lesion between the inferior and superior vena cava, making a third lesion extending laterally along the right atrium and intersecting the second lesion, making fourth lesion in the coronary sinus, making a fifth lesion extending along a transverse line located below the right and left inferior pulmonary veins, making a sixth lesion extending along a transverse line located above the right and left superior pulmonary veins, and making a seventh lesion comprising a plurality of lesions extending along the anterior interatrial groove proximate the origins of the right superior and inferior pulmonary veins and intersecting the fifth transverse lesion below the pulmonary veins and the sixth transverse lesion above the pulmonary veins, and making an eighth lesion located along a line extending from the base of the left atrial appendage to a location proximate the mitral annulus, is accomplished using a catheter system comprising an endocardial catheter and epicardial catheter. Both catheters may be configurable so that they can be shaped to correspond to the desired lesion curvature. In some embodiments, catheters are magnetized or selectively magnetizable (with electromagnets) so that they attract each other through the myocardium (the wall of the atrium). Either catheter, or both catheters, may be an ablation catheter, operable to create transmural lesions with RF energy (bipolar or monopolar), microwave, laser, or cryoablation.

In some embodiments, a lesion along the superior to inferior vena cava may be made using a catheter comprising an ablation member at or near its distal end. Ablation energy supplied by the ablation member may be of any source sufficient to damage the target tissue leading to formation of conduction-blocking scar tissue at the lesion site. For example, the source of ablation energy may be selected from the group consisting of radio frequency (RF) energy, microwave energy, thermal energy, cryogenic energy, laser energy, and high-frequency ultrasound energy. In some embodiments, the source of ablation energy is a cryogen. In several embodiments, a catheter delivers ablation energy from the endocardium transmurally to the epicardium. Optionally, formation of the lesion may be visually observed endoscopically on the epicardial surface. In some embodiments, lesion formation is visually observed via a scope placed through a subxiphoid access incision. Further optionally, a probe may be placed through a lumen in the scope such that it may be used for one or more of ablation guidance, supplying ablation energy, application of pressure between the working portion of the ablation member, temperature monitoring, and protection of tissues adjacent to the lesion site.

Several embodiments relate to systems, methods and apparatus for producing a superior to inferior vena cava lesion. In some embodiments, a probe comprising an ablation member may be used to create the lesion. In some embodiments, the probe may create a transmural lesion from the epicardium. In some embodiments, the probe may be passed through a lumen in a scope placed through a subxiphoid access incision or may be passed through a secondary access port in the thorax or abdomen. In some embodiments, the probe may create a transmural superior to inferior vena cava lesion from the endocardium by being placed through a further access point in the heart, for example an access point in the right atrial appendage using means such as a purse string suture or valved sheath to prevent or minimize the escape of blood from the beating heart.

Several embodiments relate to systems, methods and apparatus for producing a superior to inferior vena cava lesion using a clamp comprising an ablation member. In some embodiments, the clamp used to create a superior to inferior vena cava lesion is passed through an access port in the thorax. In some embodiments, the clamp is configured to comprise two opposing jaws that when actuated may open or close to apply pressure there between. One jaw of the clamp may be placed along the surface of the endocardium through a further access point in the heart, for example an access point in the right atrial appendage and optionally blood is prevented from escaping the beating heart using means such as a purse string suture. The other jaw of the clamp may remain external to the heart along the surface of the epicardium such that the wall of the heart is positioned between the jaws of the clamp and subjected to pressure when the clamp jaws are in a closed position. In one embodiment, both clamp jaws comprise an ablation member configured such that a transmural lesion may be made by application of ablation energy to both the internal and external surfaces of the heart adjacent the clamp. In another embodiment, one jaw comprises an ablation member and the other jaw comprises a temperature sensor that is configured to detect a temperature indicative of a lesion that has reached a sufficient state of transmurality. In several embodiments, the jaws of a clamp configured to generate a superior to inferior vena cava lesion may be a configured to allow for actuation independent of one another.

Optionally, in any of the aforementioned embodiments, a probe comprising an ablation member may be used to finalize the superior to inferior vena cava lesion so as to reduce the possibility of making contact of adjacent tissue, such as the phrenic nerve. In some embodiments, the probe may create the transmural lesion from the epicardium and may optionally be passed through a lumen in a scope placed through a subxiphoid access incision or may be passed through a secondary access port in the thorax or abdomen. In some embodiments, the probe may further comprise an insulation sheath or other such similar adjustable means configured to control the amount of surface area exposed on the working portion of the ablation member such that precise control of ablation lesion formation may be achieved in areas where sensitive tissue may be adjacent to the targeted lesion zone. By way of a non-limiting example, if an insulating sheath were actuated to expose only the very most tip of the ablation member, fine tuning or touching up of the superior to inferior vena cava lesion may be accomplished with increased precision in a manner analogous to the drawing of a line on paper using a marking pen and a fine-tipped pen.

Several embodiments relate to systems, methods and apparatus for creating a right-side “T” lesion roughly perpendicular to the superior to inferior vena cava lesion. The right-side “T” lesion may be created using the same or similar variety of systems and apparatus used to create the superior to inferior vena cava lesion.

In some embodiments, an endocardial catheter, comprising an ablation member at or near its distal end, may be used to conduct ablation energy to the targeted tissue to create the right-side T lesion. Ablation energy supplied by the ablation member may be of any source sufficient to damage the target tissue leading to formation of conduction-blocking scar tissue at the lesion site. For example, the source of ablation energy may be selected from the group consisting of radio frequency (RF) energy, microwave energy, thermal energy, cryogenic energy, laser energy, and high-frequency ultrasound energy. In some embodiments, the source of ablation energy is a cryogen. In some embodiments, a catheter comprising an ablation member delivers ablation energy from the endocardium transmurally to the epicardium. The catheter may optionally be configured to steer or otherwise be turned about 90 degrees to the direction to the axis of the vena cava so that it may be positioned to create a lesion across the right side of the heart transverse to the vena cava, most preferably about mid way between the superior and inferior vena cava and traversing the right side of the heart. Optionally, formation of the lesion may be visually observed endoscopically on the epicardial surface. In some embodiments, lesion formation is visually observed via a scope placed through a subxiphoid access incision. Further optionally, a probe may be placed through a lumen in the scope such that it may be used for one or more of ablation guidance, supplying ablation energy, application of pressure between the working portion of the ablation member, temperature monitoring, and protection of tissues adjacent to the lesion site.

In some embodiments, a probe comprising an ablation member may be used to create the right-side T lesion. In some embodiments, the probe may create a transmural lesion from the epicardium. In some embodiments, the probe may be passed through a lumen in a scope placed through a subxiphoid access incision or may be passed through a secondary access port in the thorax or abdomen. In some embodiments, the probe may create the transmural lesion from the endocardium by being placed through a further access point through the heart, for example, the probe may be placed through an access point in the right atrial appendage, optionally using means such as a purse string suture or valved sheath to prevent the escape of blood from the beating heart. In some embodiments, the probe may be configured to steer or be bent so that the roughly 90 degree turn from the vena cava may be accomplished. In some embodiments, the probe may be constructed of a flexible material that allows the probe to be bent to the preferred shape and then inserted into the vena cava to navigate transverse from the vena cava across the right side of the heart to make the desired lesion. In some embodiments, the probe may be pre-configured in a shape that allows the probe to be inserted into the vena cava to navigate transverse from the vena cava across the right side of the heart to make the desired lesion.

In some embodiments, a clamp comprising an ablation member may be used to create the right-side T lesion. In some embodiments, the clamp may be passed through a secondary access port in the thorax. In some embodiments, the clamp is configured to comprise two opposing jaws that when actuated may open or close to apply pressure there between. One jaw of the clamp may be placed along the surface of the endocardium through a further access point through the heart, for example through an access point in the right atrial appendage, optionally using means such as a purse string suture that may prevent the escape of blood from the beating heart. The other jaw of the clamp may remain external to the heart along the surface of the epicardium such that the wall of the heart is positioned between the jaws of the clamp and subjected to pressure when the clamp jaws are actuated closed. In one embodiment, both clamp jaws comprise an ablation member and are configured such that a transmural lesion may be made from both the internal and external surfaces of the heart adjacent the clamp. In another embodiment, the clamp is configured such that one jaw comprises an ablation member and the other jaw comprises a temperature sensor that is configured to detect a temperature indicative of a lesion that has reached a sufficient state of transmurality. In some embodiments, the jaws of a clamp configured to create the right-side T lesion may be a configured to allow for actuation independent of one another. The clamp may optionally be configured to steer or be bent so that the right-side T lesion may be made at about 90 degrees from the point of access. In some embodiments, the clamp may be constructed of a flexible material that allows the clamp to be bent to the preferred shape and then inserted and positioned across the right side of the heart to make the desired lesion. In some embodiments, the clamp may pre-configured in the desired shape to create the right-side T lesion.

Several embodiments relate to systems, methods and apparatus for creating a right-side lateral lesion roughly parallel to the superior to inferior vena cava. The right-side lateral lesion may be created using the same or similar variety of systems and apparatus used to create the superior to inferior vena cava lesion.

In some embodiments, an endocardial catheter comprising an ablation member at or near its distal end, may be used to conduct ablation energy to the targeted tissue to create the right-side lateral lesion. Ablation energy supplied by the ablation member may be of any source sufficient to damage the target tissue leading to formation of conduction-blocking scar tissue at the lesion site. For example, the source of ablation energy may be selected from the group consisting of radio frequency (RF) energy, microwave energy, thermal energy, cryogenic energy, laser energy, and high-frequency ultrasound energy. In some embodiments, the source of ablation energy is a cryogen. In some embodiments, a catheter comprising an ablation member delivers ablation energy from the endocardium transmurally to the epicardium. In some embodiments, the catheter may be configured to steer or otherwise be turned about 180 degrees to the direction to the axis of the vena cava so that it may be positioned to create a lesion extending roughly perpendicular from the right-side T and terminating in proximity to the right atrial appendage. Optionally, formation of the lesion may be visually observed endoscopically on the epicardial surface. In some embodiments, lesion formation is visually observed via a scope placed through a subxiphoid access incision. Further optionally, a probe may be placed through a lumen in the scope such that it may be used for one or more of ablation guidance, supplying ablation energy, application of pressure between the working portion of the ablation member, temperature monitoring, and protection of tissues adjacent to the lesion site.

In some embodiments, a probe comprising an ablation member may be used to create the right-side lateral lesion. In some embodiments, the probe may create a transmural lesion from the epicardium. In some embodiments, the probe may be passed through a lumen in a scope placed through a subxiphoid access incision or may be passed through a secondary access port in the thorax or abdomen. In some embodiments, the probe may create the transmural lesion from the endocardium by being placed through a further access point through the heart, for example, the probe may be placed through an access point in the right atrial appendage, optionally using means such as a purse string suture or valved sheath to prevent the escape of blood from the beating heart. The probe may be configured to steer or be bent so that the roughly 180 degree turn from the vena cava may be accomplished. In some embodiments, the probe may be constructed of a flexible material that allows the probe to be bent to the preferred shape and then inserted into the vena cava to navigate transverse from the vena cava across the right side of the heart and about 180 degrees to make the desired lesion vertically along the right atrium. In some embodiments, the probe may be preconfigured in the desired shape and then inserted into the vena cava to navigate transverse from the vena cava across the right side of the heart and about 180 degrees to make the desired lesion vertically along the right atrium.

In some embodiments, a clamp comprising an ablation member may be used to create the right-side lateral lesion. In some embodiments, a clamp configured to create the right-side lateral lesion may be passed through a secondary access port in the thorax. In some embodiments, the clamp is configured to have two opposing jaws that when actuated may open or close to apply pressure there between. One jaw of the clamp may be placed along the surface of the endocardium through a further access point through the heart, for example through an access point in the right atrial appendage, optionally using means such as a purse string suture to prevent the escape of blood from the beating heart. The other jaw of the clamp may remain external to the heart along the surface of the epicardium such that the wall of the heart is positioned between the jaws of the clamp and subjected to pressure when the clamp jaws are actuated closed. In one embodiment, both clamp jaws comprise an ablation member configured such that a transmural lesion may be made from both the internal and external surfaces of the heart adjacent the clamp. In another embodiment one jaw comprises an ablation member and the other jaw comprises a temperature sensor configured to detect a temperature indicative of a lesion that has reached a sufficient state of transmurality. In some embodiments, the jaws of a clamp configured to create the right-side lateral lesion may be configured to allow for actuation independent of one another. The clamp may optionally be configured to steer or be bent so that the right-side lateral lesion may be made at about 180 degrees from the point of access. In some embodiments, the clamp may be constructed of a flexible material that may allow the clamp to be bent to the preferred shape and then inserted and positioned across the right side of the heart to make the right-side lateral lesion. In some embodiments, the clamp pre-configured in the desired shape to make the right-side lateral lesion.

Several embodiments relate to systems, methods and apparatus for placing a lesion inside the coronary sinus. In some embodiments, a catheter comprising an ablation means at its distal end may be used to create a lesion inside the coronary sinus. In some embodiments, catheter access may be through the vena cava or other such suitable route amenable to catheter navigation. Ablation energy supplied by the ablation member may be of any source sufficient to damage the target tissue leading to formation of conduction-blocking scar tissue at the lesion site. For example, the source of ablation energy may be selected from the group consisting of radio frequency (RF) energy, microwave energy, thermal energy, cryogenic energy, laser energy, and high-frequency ultrasound energy. In some embodiments, the source of ablation energy is a cryogen. The ablation member may be further configured to comprise an expanding member that may be expanded to contact the inner lumen of the coronary sinus. The expanding member may be in any form sufficient to contact and conform to the shape of the coronary sinus inner lumen. In some embodiments, the expanding member may be an inflatable balloon configured to transmit the ablative energy for the creation of a lesion in the coronary sinus. In other embodiments, the expanding member may be an expandable framework, such as a basket or cage, configured to transmit the ablative energy for the creation of a lesion in the coronary sinus. The ablation member may be of any length suitable for sufficient ablative energy transfer. In some embodiments, the ablation member may be of a length that minimizes the number of ablation cycles necessary to form a lesion of sufficient surface area to block macro-reentrant circuits. Optionally, formation of the lesion may be visually observed endoscopically on the epicardial surface. In some embodiments, lesion formation is visually observed via a scope placed through a subxiphoid access incision.

In some embodiments, a probe comprising an ablation member may be used for the creation of a lesion in the coronary sinus. In some embodiments, the probe may create the lesion by being placed through an access point in the heart, for example, the probe may be placed through an access point in the right atrial appendage, optionally using means such as a purse string suture or valved sheath to prevent the escape of blood from the beating heart. The probe may be configured to comprise an expanding structure such as a balloon, a basket, a coil, a loop, or the like, that is configured to deliver ablation energy of the types described herein to the targeted tissue to create a lesion in the coronary sinus. In some embodiments, the probe is configured to comprise an expanding structure such as a balloon, a basket, a coil, a loop, or the like, that is configured to deliver a cryogen ablative energy source to the targeted tissue to create a lesion in the coronary sinus.

In the embodiments described herein, left-side lesions may be formed using a variety of surgical and electrophysiological tools. In some embodiments, access is gained to the heart for creating left-side lesions through a small thorocotomy incision located at an interstitial location between the left-side rib bones of the chest.

In some embodiments, lesions are placed traversing the left side of the heart, with one lesion traversing a path extending across the left and right inferior pulmonary veins, and a second lesion traversing a path extending across the left and right superior pulmonary veins (the “PV lesions”). In some embodiments, the PV lesions intersect at a point in proximity to the left atrial appendage and then diverge along a superior and inferior path of traverse. In some embodiments, an additional lesion may be placed to intersect the PV lesions at a point in proximity to the left atrial appendage. An ablation member may be used to conduct ablation energy to the targeted tissue to create the PV lesions. Ablation energy supplied by the ablation member may be of any source sufficient to damage the target tissue leading to formation of conduction-blocking scar tissue at the lesion site. For example, the source of ablation energy may be selected from the group consisting of radio frequency (RF) energy, microwave energy, thermal energy, cryogenic energy, laser energy, and high-frequency ultrasound energy. In some embodiments, the source of ablation energy is a cryogen.

In some embodiments, the PV lesions may be formed using a clamp comprising an ablation member. In some embodiments, a clamp configured to create the PV lesions may be passed through a left-side thorocotomy. The clamp may be configured to comprise two opposing jaws that when actuated may open or close to apply pressure there between. One jaw of the clamp may be placed along the surface of the endocardium through a further access point through the heart, for example the clamp may be placed through an access point in the left atrial appendage, optionally using means such as a purse string suture to prevent the escape of blood from the beating heart. The other jaw of the clamp may remain external to the heart along the surface of the epicardium such that the wall of the heart is positioned between the jaws of the clamp and subjected to pressure when the clamp jaws are actuated closed. In one embodiment, both clamp jaws comprise an ablation member such that a transmural lesion may be made from both the internal and external surfaces of the heart adjacent the clamp. In another embodiment one jaw comprises an ablation member and the other jaw comprises a temperature sensor configured to detect a temperature indicative of a lesion that has reached a sufficient state of transmurality. In some embodiments, the jaws of the clamp configured to create the PV lesions may be configured to allow for actuation independent of one another. Optionally, the jaws of the clamp may further comprise magnets that contribute to the clamping pressure such that lesion formation may be aided by the additional pressure.

In some embodiments, a probe comprising an ablation means may be used for formation of the PV lesions. In some embodiments, the probe may create a transmural lesion from the endocardium. In some embodiments, the probe may be placed through an access point through the heart, for example, the probe may be placed through an access point in the left atrial appendage, optionally using means such as a purse string suture or valved sheath that may prevent the escape of blood from the beating heart.

Optionally, formation of the lesion may be visually observed endoscopically on the epicardial surface. In some embodiments, lesion formation is visually observed via a scope placed through a subxiphoid access incision. Further optionally, a probe may be placed through a lumen in the scope such that it may be used for one or more of ablation guidance, supplying ablation energy, application of pressure between the working portion of the ablation member, temperature monitoring, and protection of tissues adjacent to the lesion site.

In several embodiments described herein, the right pulmonary veins are further isolated by forming lesions that close off the divergent portion of the PV lesions (the “RPV lesions”). An ablation member may be used to conduct ablation energy to the targeted tissue to create the RPV lesions. Ablation energy supplied by the ablation member may be of any source sufficient to damage the target tissue leading to formation of conduction-blocking scar tissue at the lesion site. For example, the source of ablation energy may be selected from the group consisting of radio frequency (RF) energy, microwave energy, thermal energy, cryogenic energy, laser energy, and high-frequency ultrasound energy. In some embodiments, the source of ablation energy is a cryogen.

In some embodiments, a probe comprising an ablation member at its distal end may be placed through a lumen of an endoscope placed through a subxiphoid access point and used for the formation of the RPV lesions. In some embodiments, the probe is positioned on the epicardium proximate the anterior interatrial groove near the origin of the right pulmonary veins such that a lesion may be created along the perimeter of the pulmonary veins to complete the RPV lesions, thereby preferably forming a contiguous lesion extending from a point proximate the left atrial appendage which traverses superior and inferior to the pulmonary veins and which forms a closed loop along the origins of the right pulmonary veins.

In some embodiments, a balloon catheter may be positioned and inflated to expand the ostium of each of the right pulmonary veins so as to temporarily diminish the heat sink effect of cavitary blood passing through the vein in proximity to the RPV lesion as it is being formed. Secondarily, the resultant expansion of the ostium from the inflation of the balloon may expose a larger and more accessible surface area of the epicardium where the probe is placed for RPV lesion formation. Any acceptable means for catheter access may be used. In some embodiments, access is gained through the left atrial appendage using means such as a purse string suture or valved sheath to prevent the escape of blood from the beating heart.

In some embodiments for forming the RPV lesion, a probe may be configured to comprise a shaped end that facilitates the shaping of the RPV lesion from the endocardium. In some embodiments, the probe distal portion may comprise a loop-like feature or plurality of loop-like features to aid in providing the desired contact pressure against the endocardium. In some embodiments, the probe distal end may be exposed from a sheath such that the loop-like feature or features are unconstrained and allowed to be formed by mechanical action or thermal action if a shape memory alloy is used. In the instance of a single loop-like feature, the feature provides locating force against the endocardium and also provides the working ablative surface for lesion formation. In the instance of a plurality of loop-like features, one or more features may be used for locating and securement while one or more features may be used for ablation. Hooks, barbs, or other such means may be further used to aid in securement in any endocardial probe embodiment.

In several embodiments described herein, a lesion is formed along the left atrial appendage. In some embodiments, an ablation probe comprising an ablation member at its distal portion is placed on the surface of the endocardium to form the lesion. Ablation energy supplied by the ablation member may be of any source sufficient to damage the target tissue leading to formation of conduction-blocking scar tissue at the lesion site. For example, the source of ablation energy may be selected from the group consisting of radio frequency (RF) energy, microwave energy, thermal energy, cryogenic energy, laser energy, and high-frequency ultrasound energy. In some embodiments, the source of ablation energy is a cryogen. The lesion may serve to isolate the electrical path along the left atrial appendage.

In several embodiments for forming the left atrial appendage lesion, the probe may either be steerable or curved to conform along the left atrial appendage access point to the mitral annulus. In some embodiments, the probe may be configured to steer or be bent so that a roughly 180 degree turn may be accomplished. In some embodiments, the probe may be constructed of a flexible material that allows the probe to be bent to the preferred shape prior to insertion by either manually forming the desired bend or by having a bend that increases as the probe tip is unrestrained from a sheath. In some embodiments, the probe tip may be steered by means that are controlled from the distal end by the operator.

Optionally, for any portion of the procedure, pericardium may be insufflated with a gas or biocompatible fluid such that the pericardium is lifted away from the epicardium to improve the viewing of lesion formation when observed by endoscope.

Several embodiments relate to a catheter comprising an ablation member at its distal end comprised of an ablation surface with an ablation energy source providing energy to the ablation surface. The ablation energy may be of any type sufficient to damage the target tissue leading to formation of conduction-blocking scar tissue at the lesion site. For example, the source of ablation energy may be selected from the group consisting of radio frequency (RF) energy, microwave energy, thermal energy, cryogenic energy, laser energy, and high-frequency ultrasound energy. In some embodiments, the source of ablation energy is a cryogen.

In some embodiments, the distal portion of the catheter may further comprise an expanding structure that comprises the ablation surface or a plurality of ablation surfaces. In some embodiments, the expanding structure may expand by thermal action, such as by use of shape memory materials, or may be mechanically actuated. In some embodiments, the expandable structure may be comprised of any of a balloon, one or more of coils or loops, a basket, a cage, a flange or bell-like structure and the like.

Several embodiments described herein relate to a cryosurgical clamp comprising an ablation member configured to create ablation lesions leading to formation of conduction-blocking scar tissue at the lesion site. In some embodiments, the clamp is configured to have two opposing jaws that when actuated may open or close to apply pressure there between.

In some embodiments, one or more jaws of the clamp are configured to comprise an ablation member configured to conduct ablation energy to the targeted tissue to create the desired lesion. Ablation energy supplied by the ablation member may be of any source sufficient to damage the target tissue leading to formation of conduction-blocking scar tissue at the lesion site. For example, the source of ablation energy may be selected from the group consisting of radio frequency (RF) energy, microwave energy, thermal energy, cryogenic energy, laser energy, and high-frequency ultrasound energy. In some embodiments, the source of ablation energy is a cryogen.

In some embodiments, the cryosurgical clamp may have a thin shaft so that it may be introduced though a very small opening, such as that of a mini-thorocotomy in the chest wall or an endoscope. For example, when closed, the clamp may be inserted through a very small chest wall incision. After it is positioned inside the chest, the clamp jaws may be opened wide enough to preferably be able to clamp large structures. In some embodiments, the clamp may be manipulated by the clamp's handle which is well outside the chest.

In some embodiments, the clamp may be bipolar, having an ablative surface on the opposing surfaces of the two jaws. Alternately, the clamp may be monopolar with an ablative surface on one jaw. In some embodiments, the clamp may be configured such that one or both jaws further comprise a temperature sensor that is configured to detect a temperature indicative of a lesion that has reached a sufficient state of transmurality.

In several embodiments described herein, a steerable cryoprobe may be used to perform one or more of the lesions of the procedure described herein. In some embodiments, the probe may be comprised of an ablation surface at its distal portion. In some embodiments, the probe may further comprise a retractable sheath or shaft which surrounds the ablation surface. In some embodiments, the ablation surface is sized to provide a desirable combination of access size, stiffness, and working surface area. In some embodiments, the inner cryoprobe may be freely moveable through the handle and shaft so that it can be lengthened or withdrawn completely inside the shaft.

In some embodiments, the shaft of the instrument may provide sufficient stiffness to provide strength when pressure is applied for surface contact during lesion formation while remaining malleable so that it can be shaped. In one embodiment, steering or shaping may be performed by hand before insertion and use.

### BRIEF DESCRIPTION OF THE DRAWINGS

FIG. 1A shows the Maze VII lesion pattern.

FIG. 1B shows schematic views of the human heart depicting the effect of the Maze VII lesion pattern on macro re-entrant circuits.

FIG. 2 shows a schematic representation of the cycle of normal sinus rhythm (NSR), trigger and macro re-entrant circuits underlying intermittent atrial fibrillation (AF).

FIG. 3 is a schematic diagram of a view of the human heart showing the SVC-IVC Maze VII lesion, which extends between the superior vena cava (SVC) and inferior vena cava (IVC).

FIG. 4 shows a front view of the human heart with the right ventricle opened depicting one embodiment for creating the Maze VII SVC-IVC lesion.

FIG. 5 shows a front view of the human heart with the right ventricle opened depicting one embodiment for touching up the Maze VII SVC-IVC lesion for completeness.

FIG. 6 shows a front view of the human heart with the right ventricle opened depicting one the completed SVC-IVC Maze VII lesion.

FIG. 7 is a schematic diagram of a view of the human heart showing the location of the “T” lesion in relation to the SVC-IVC lesion.

FIG. 8 shows a front view of the human heart with the right ventricle opened depicting a schematic representation of a physician viewing the “T” lesion.

FIG. 9 is a schematic diagram of a view of the human heart showing the location of the RA lateral lesion in relation to the SVC-IVC and T lesions.

FIG. 10 shows a front view of the human heart with the right ventricle opened depicting one embodiment for creating the Maze VII RA lateral lesion.

FIG. 11 shows a front view of the human heart with the right ventricle opened depicting a schematic representation of a physician viewing the RA lateral lesion in relation to the SVC-IVC and T lesions.

FIG. 12 is a schematic diagram of a view of the human heart showing the location of the Coronary Sinus lesion in relation to the relation to the SVC-IVC lesion, the T lesion and the RA lateral lesion.

FIG. 13 shows a view of the opened right atrium of the human heart depicting one embodiment for creating the Maze VII Coronary Sinus lesion.

FIG. 14 shows a surface view of the human heart depicting one embodiment for creating the Maze VII Coronary Sinus lesion in relation to the SVC-IVC and T lesions.

FIG. 15 shows a surface view of the human heart indicating the locations of the SVC-IVC and T lesions and depicting one embodiment for creating the Maze VII Coronary Sinus lesion.

FIG. 16 is a schematic diagram of a view of the human heart showing the location of the Inferior LA lesion in relation to the Coronary Sinus, SVC-IVC, T and the RA lateral lesions.

FIG. 17 shows a surface view of the human heart depicting placement of a purse string suture (dashed line) and security loop in one embodiment for accessing the left atrial appendage (LAA) as part of the Maze VII procedure.

FIG. 18 shows a surface view of the human heart depicting the positioning of a cryosurgical clamp in one embodiment for creating the Maze VII Inferior left atrium (LA) lesion.

FIG. 19 shows a surface view of the human heart depicting one embodiment for creating the Maze VII Inferior LA lesion.

FIG. 20 shows a surface view of the human heart depicting a completed Inferior LA lesion in relation to the Coronary Sinus, SVC-IVC, T and the RA lateral lesions in a partially completed Maze VII procedure.

FIG. 21 is a schematic diagram of a view of the human heart showing the location of the Superior LA lesion in relation to the Inferior LA, Coronary Sinus, SVC-IVC, T and RA lateral lesions.

FIG. 22 shows a surface view of the human heart depicting one embodiment for creating the Maze VII Superior LA lesion.

FIG. 23 shows a surface view of the human heart depicting a completed Superior LA lesion in relation to the Inferior LA, Coronary Sinus, SVC-IVC, T and RA lateral lesions in a partially completed Maze VII procedure.

FIG. 24 is a cross-sectional view of the left atrium and ventricle of the human heart showing one embodiment for completing lesions to isolate the Pulmonary Veins in the Maze VII procedure.

FIG. 25A shows a surface view of the human heart depicting one embodiment for completing lesions to isolate the Pulmonary Veins in the Maze VII procedure.

FIG. 25B is a schematic diagram of a view of the human heart showing the location of the right PV lesion across the ostia of the right pulmonary veins in relation to the Superior and Inferior LA lesions.

FIG. 26 is a schematic diagram of a view of the human heart showing the location of the Sub-LAA lesion.

FIG. 27 is a cross-sectional view of the left atrium and ventricle of the human heart showing one embodiment for creating the Sub-LAA lesion of the Maze VII procedure.

FIG. 28A is a cross-sectional view of the left atrium and ventricle of the human heart showing one embodiment for creating the Sub-LAA lesion of the Maze VII procedure.

FIG. 28B is a cross-sectional view of the anatomy of the human heart in proximity to the Sub-LAA lesion showing the short distance between the base of the LAA and the Mitral Annulus.

FIG. 28C is a surface view showing the anatomy of the human heart in proximity to the Sub-LAA lesion.

FIG. 28D is a cross-sectional view of the left atrium and ventricle of the human heart showing one embodiment for closing the LAA access point.

FIG. 29 shows a schematic representation of examples of lesions formed by various energy delivery sources.

FIG. 30 shows a schematic representation of visualization of “iceball” formation during cryosurgery.

FIG. 31 shows a schematic representation comparing lesions formed by various energy delivery sources.

FIG. 32 shows one embodiment of a bipolar ablation clamp.

FIG. 33 shows a cross-sectional view of a portion of one jaw of the ablation clamp of FIG. 32.

FIG. 34 shows a schematic representation of the transmural delivery of cryogenic ablation energy using a bipolar cryosurgical clamp.

FIG. 35 shows one embodiment of a unipolar ablation clamp.

FIG. 36 shows a schematic representation of the transmural delivery of cryogenic ablation energy using a unipolar cryosurgical clamp.

FIG. 37 shows a schematic representation of one embodiment of a steerable ablation probe.

FIG. 38 shows a schematic representation of the ablation probe of FIG. 37 with the tip extended.

FIG. 39 shows a schematic representation of the use of a subxiphoid scope in one embodiment of the Maze VII procedure.

FIG. 40 shows a schematic representation of one embodiment of an ablation catheter with a coiled tip for ablation energy delivery. As the sheath covering the ablation member is retracted, the coiled portion of the ablation member contacts the walls of the coronary sinus to deliver ablation energy.

FIG. 41A shows a schematic representation of one embodiment of an ablation catheter with a basket tip for ablation energy delivery. As the sheath covering the ablation member is retracted, the basket tip of the ablation member expands to contact the walls of the coronary sinus to deliver ablation energy.

FIG. 41B shows a schematic representation of one embodiment of an ablation catheter with a basket tip for ablation energy delivery. As the sheath covering the ablation member is retracted, the basket tip of the ablation member expands to contact the walls of the coronary sinus.

FIG. 42 shows a schematic representation of one embodiment of an ablation catheter with a flanged tip for ablation energy delivery. As the sheath covering the ablation member is retracted, the flanged tip of the ablation member contacts the walls of the coronary sinus to deliver ablation energy.

FIG. 43 shows a schematic representation of one embodiment of an ablation catheter with a loop-like tip for ablation energy delivery.

### DETAILED DESCRIPTION

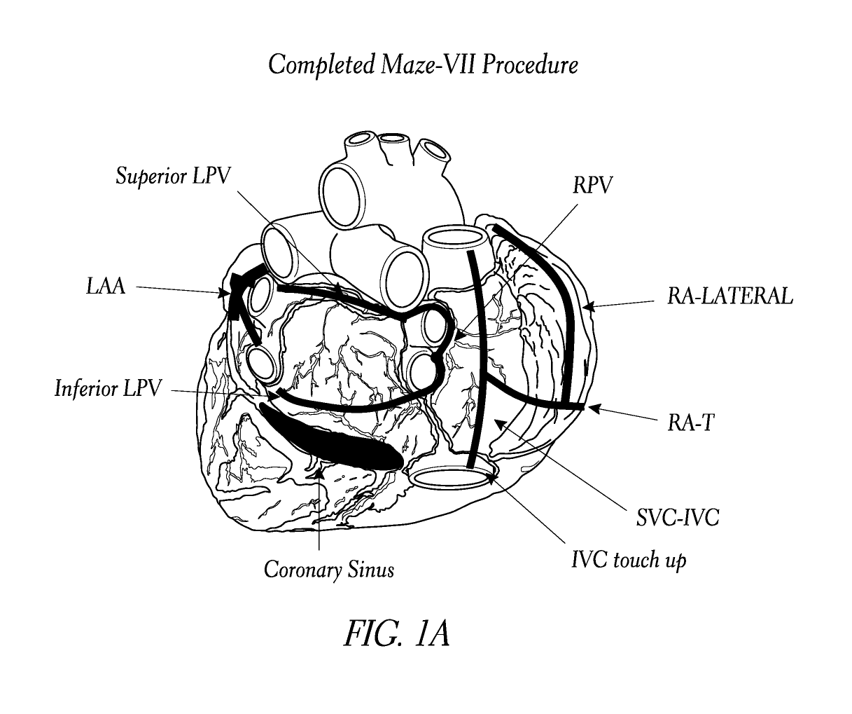

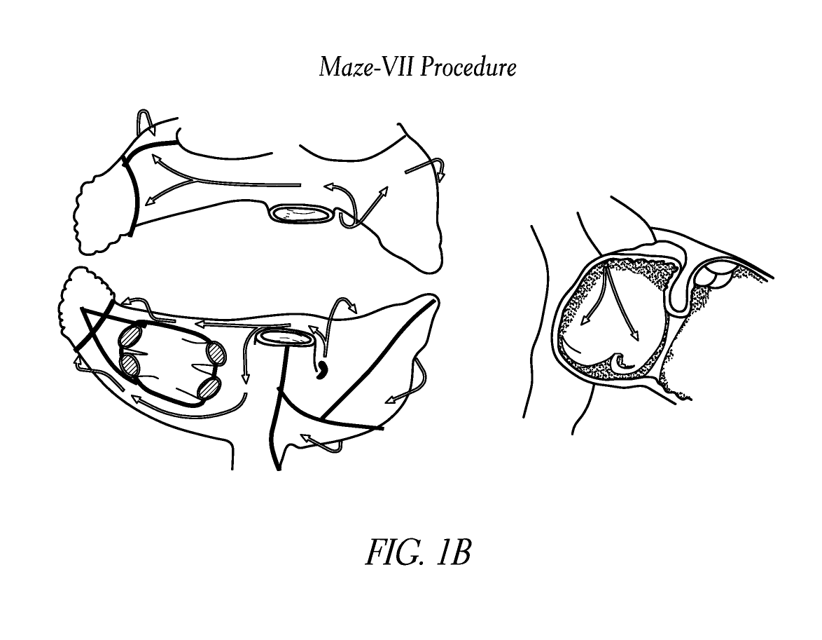

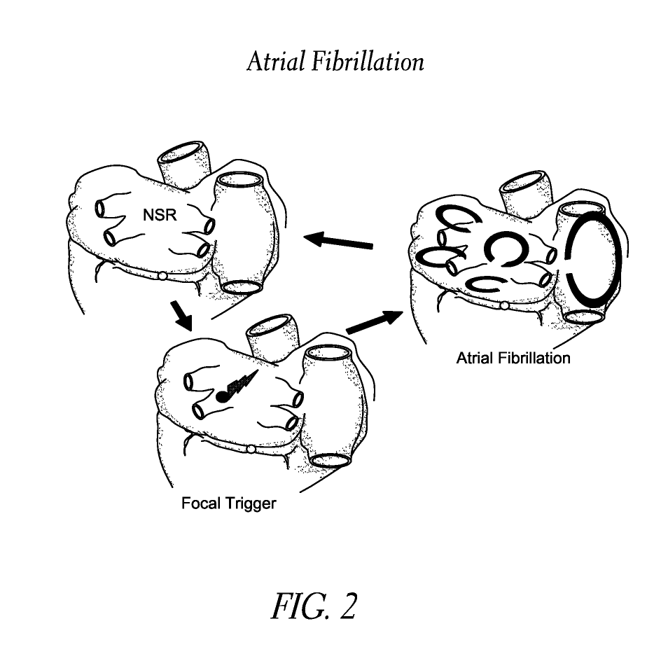

Interventional techniques which preclude development of the macro-reentrant circuits that characterize AF (See, e.g., FIG. 2) can be used to cure AF. One way that the development of macro-reentrant circuits responsible for maintaining AF can be disrupted is by placing linear lesions on the atria close enough together so that macro-reentrant circuits cannot form between them. For example, AF can be cured by “breadloafing” the atria into multiple parallel slices like a loaf of bread; however, after such procedure the atrium would be incapable of functioning properly because only the slice of the atrium containing the Sinoatrial (“SA”) node would be activated to contract. Although any pattern of linear lesions placed close enough together may be capable of curing AF, a maze pattern of lesions not only ablates AF, but also leaves the atrium capable of having a normal sinus rhythm afterwards. See, e.g., FIG. 1B. Thus, the objective of interventional therapy is to place linear lesions in such a pattern that they may not only cure the AF but may also leave the atrium capable of having a normal sinus rhythm (NSR) generated from the SA node afterward.

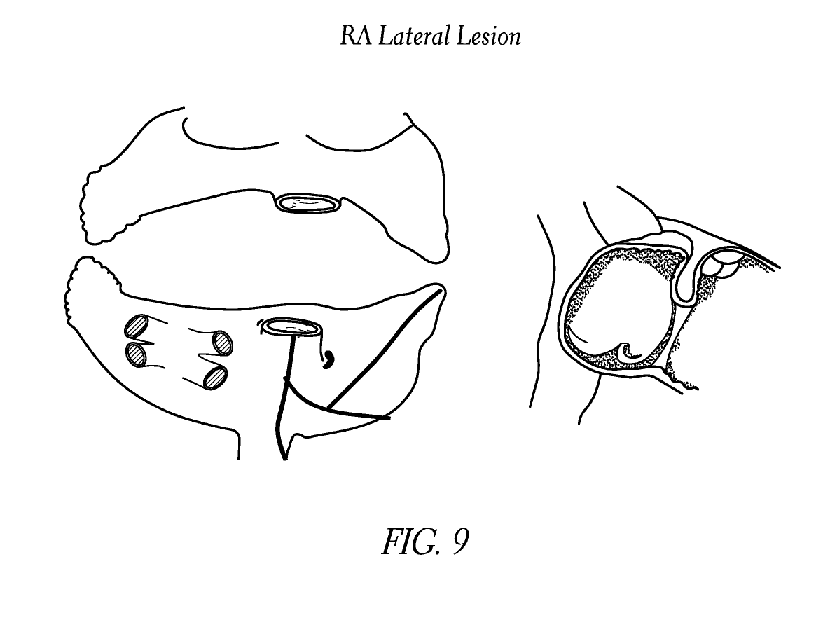

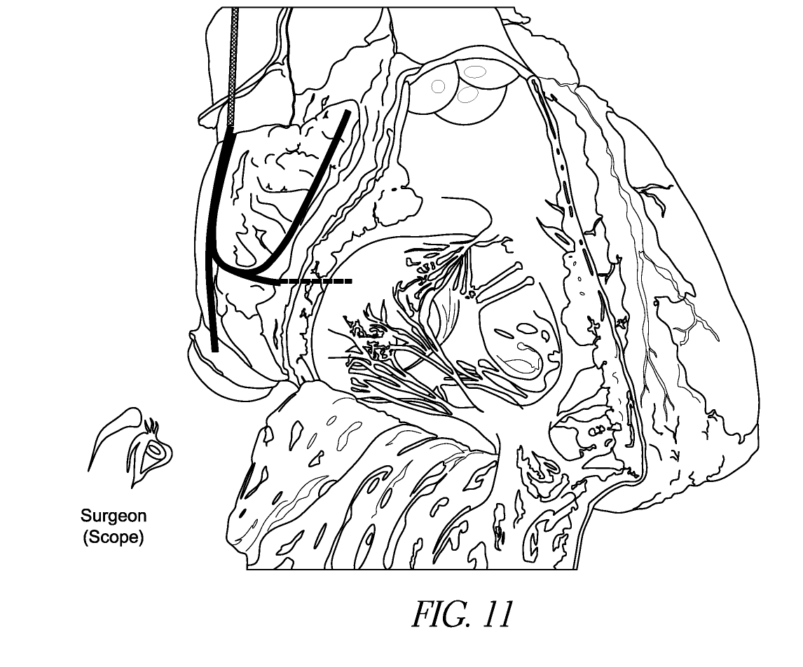

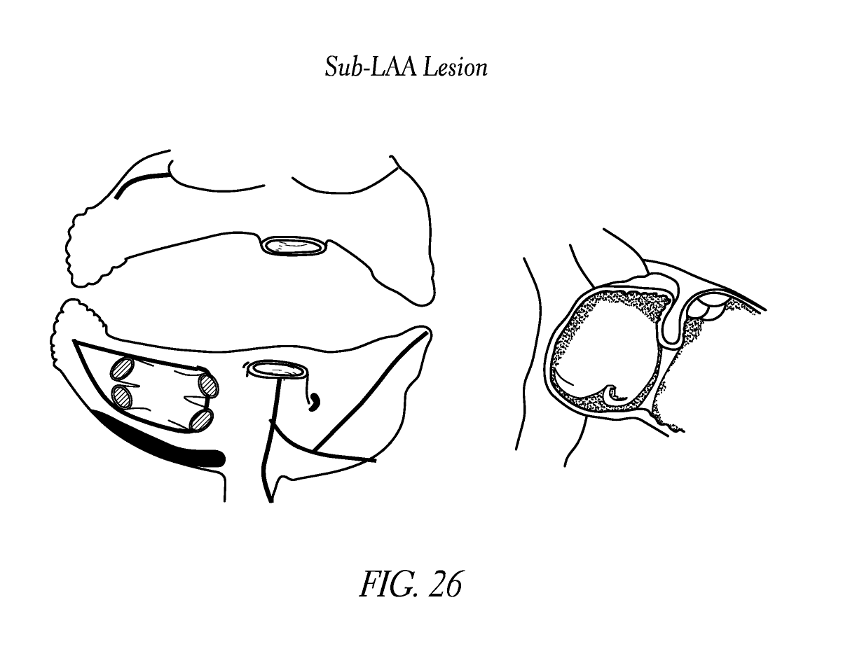

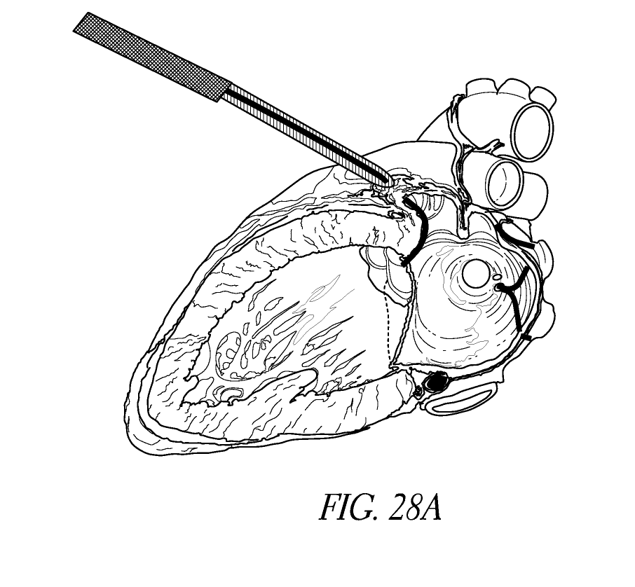

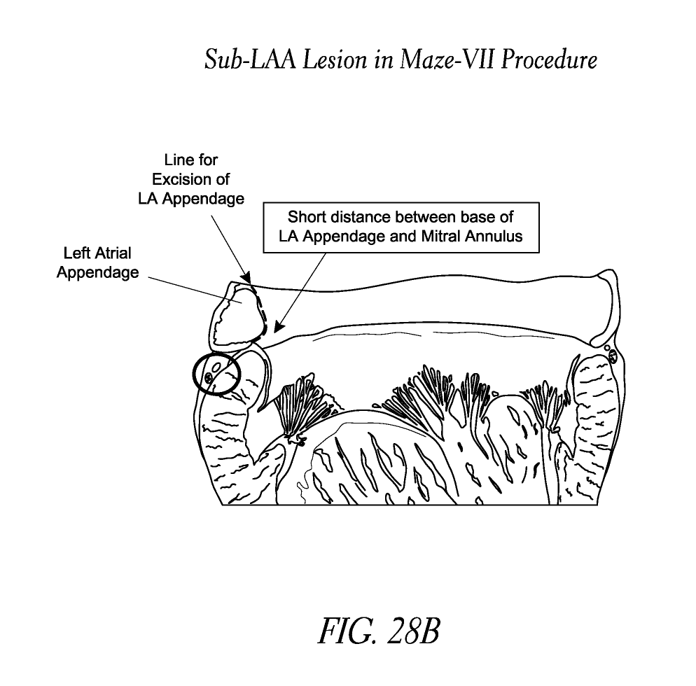

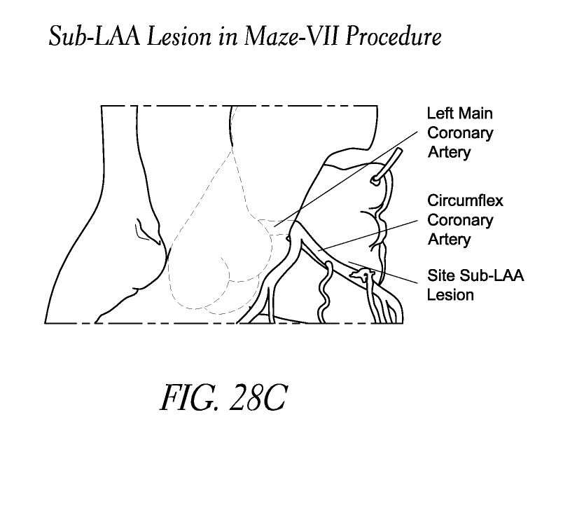

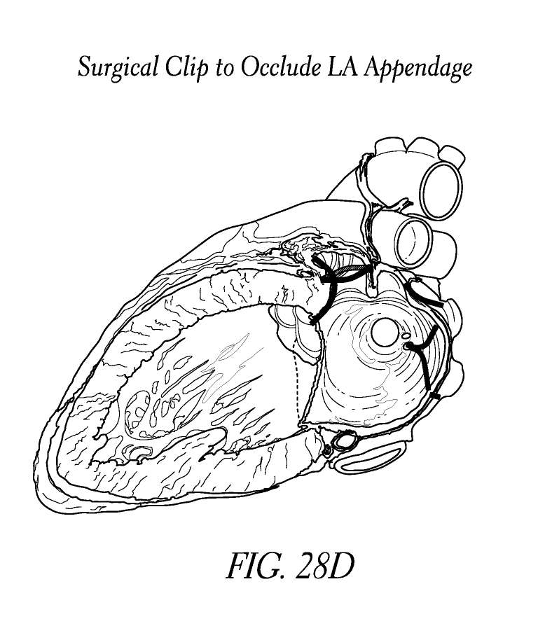

The embodiments described herein accomplish both goals of curing AF and preserving normal sinus rhythm by placing linear lesions in the pattern of a maze, such as the lesions of the Maze-VII procedure shown in FIG. 1A. The mitral line and accompanying coronary sinus lesion below the inferior pulmonary veins in the left atrium of the gold standard Cox Maze III procedure have been eliminated in the Maze-VII procedure and replaced by a single lesion beneath the overhanging left atrial appendage and approximately 1.5 cm lateral to the Left Main coronary artery, the Sub-LAA lesion (See FIG. 26 and FIG. 28). At this site, the coronary sinus has not yet formed. In addition, the Sub-LAA lesion does not affect conduction in Bachmann's Bundle. In some embodiments, the “counterlesion” that was not present in Maze-I but was added to all subsequent iterations of the Maze procedure to prevent a potential macro-reentrant circuit around the base of the right atrial appendage, is eliminated in the in the Maze-VII procedure by extending the right atrial lateral lesion from the tip of the right atrial appendage (RAA) down to the “T” lesion on the lateral right atrium (RA) (See FIGS. 9 and 11).

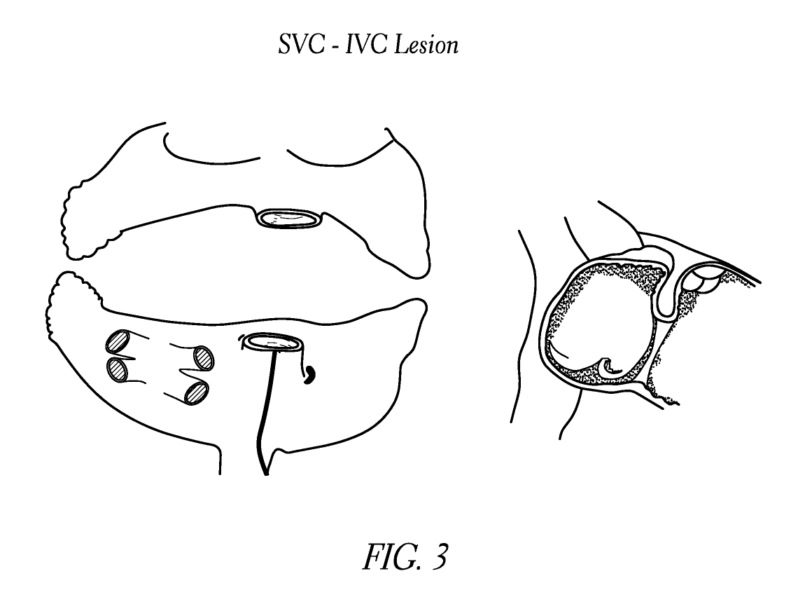

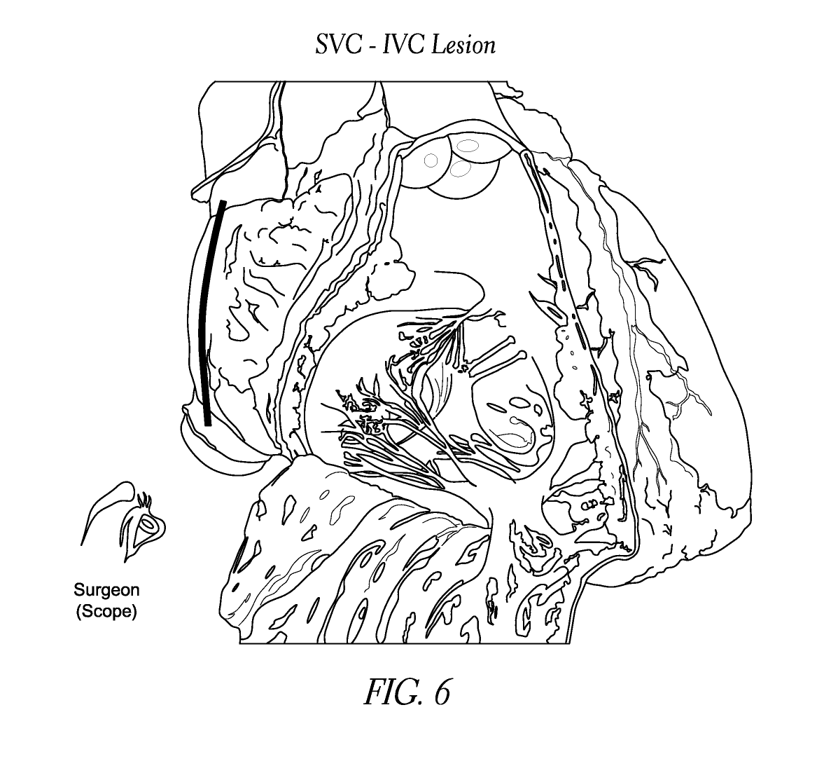

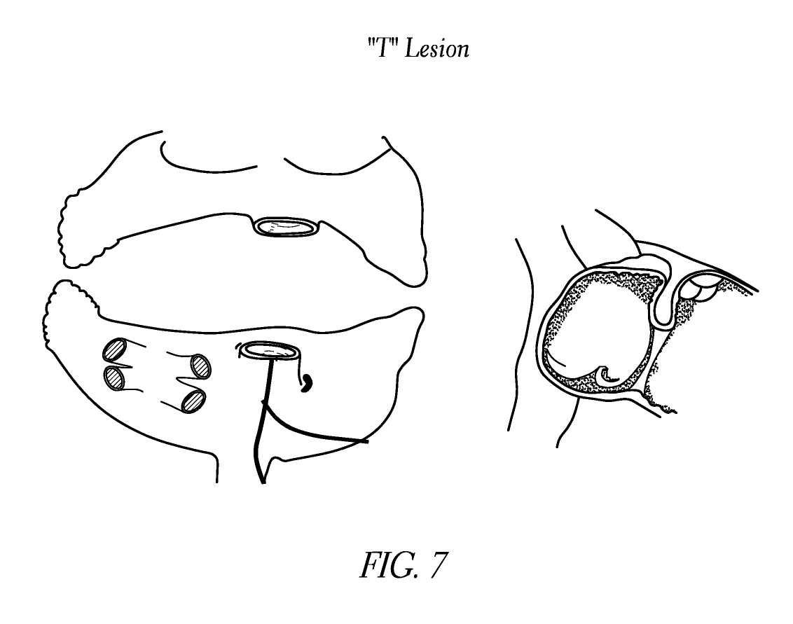

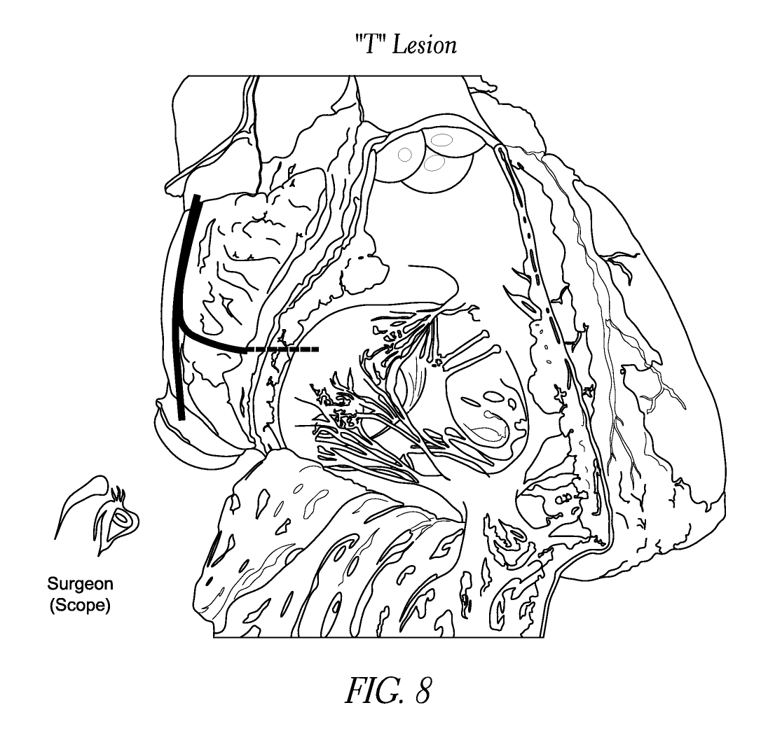

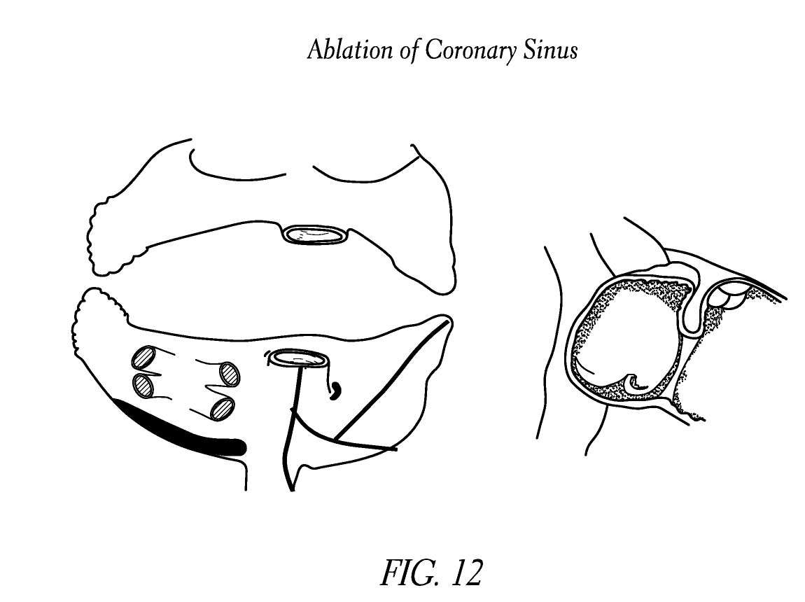

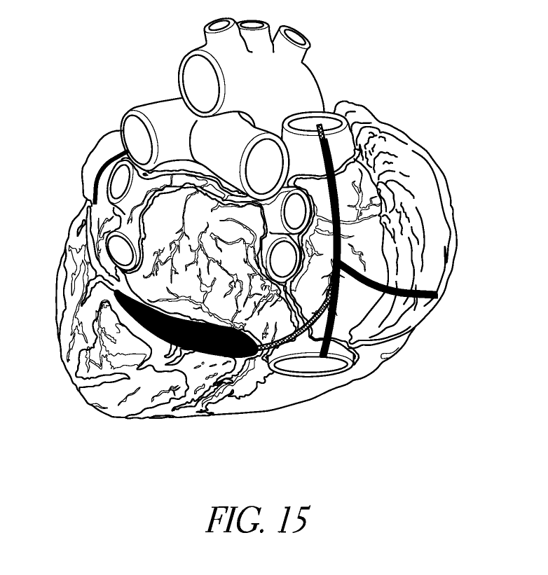

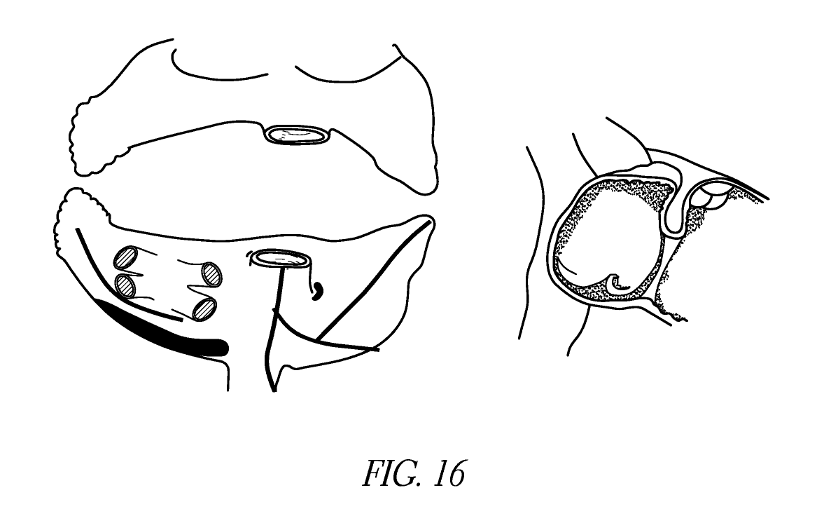

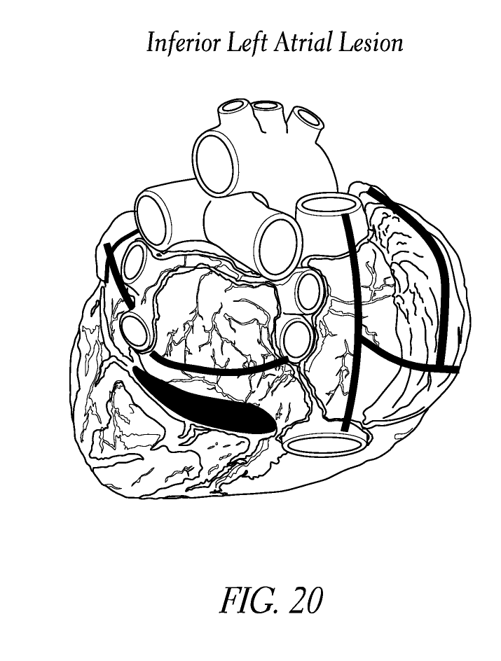

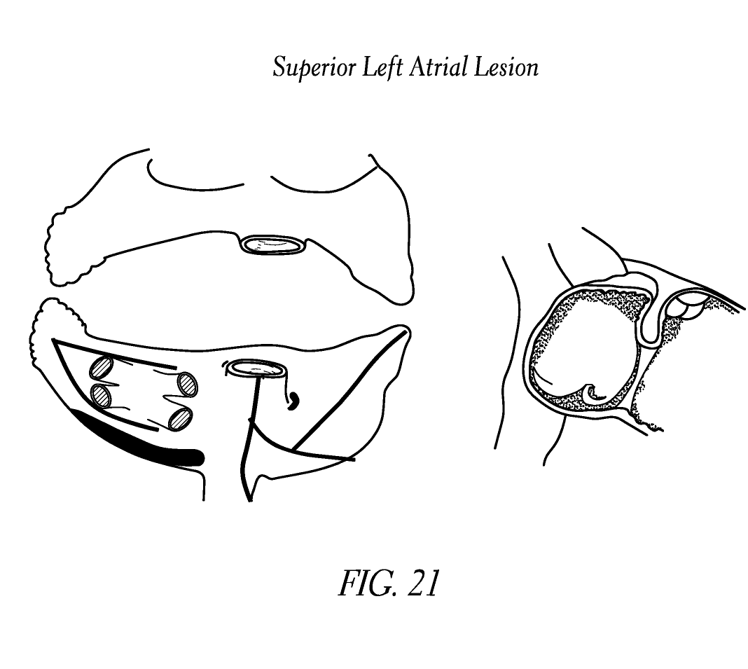

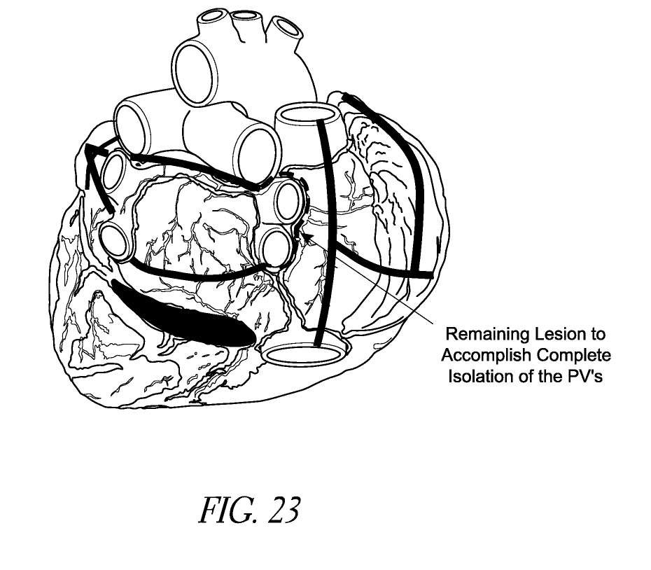

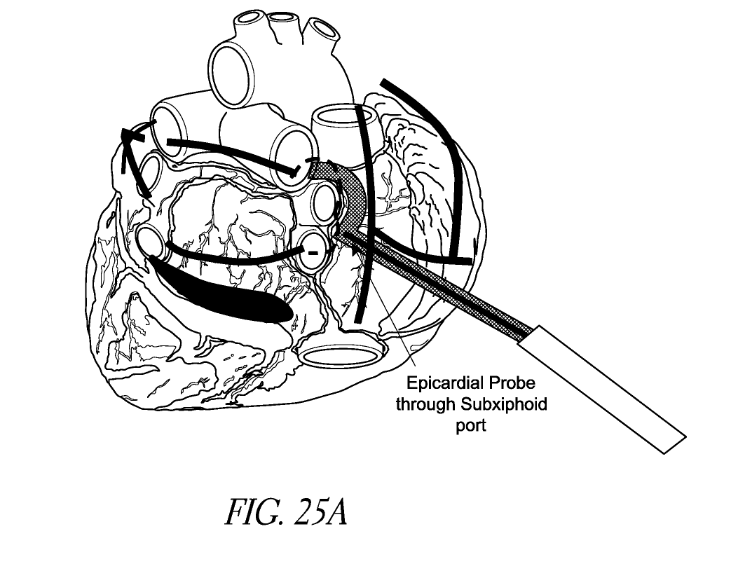

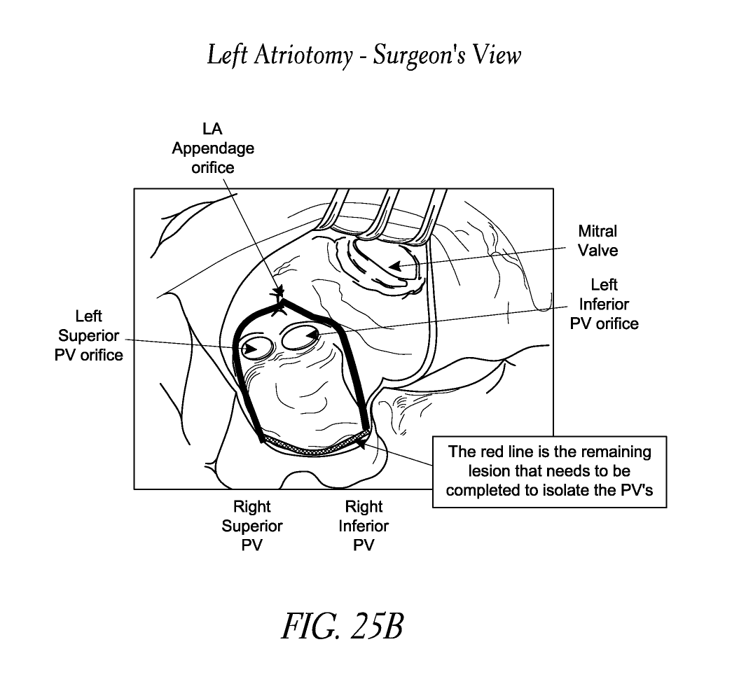

Several embodiments described herein relate to a minimally invasive interventional procedure, comprising a pattern of conduction-blocking lesions in the heart that is effective for the treatment of all forms of AF. The pattern of lesions creates a planned “maze” of scar tissue that serves as barriers, blocking the formation of aberrant macro-reentry circuits and guiding irregular cardiac electrical signals back to more normal pathways. In some embodiments, the pattern of pattern of conduction-blocking lesions comprises a first conduction-blocking lesion extending along a line between the inferior and superior vena cava (See, e.g., FIG. 1A SVC-IVC, FIG. 3 and FIG. 6), a second conduction-blocking lesion extending transversely across the right atrium and intersecting the first conduction-blocking lesion between the inferior and superior vena cava (See, e.g., FIG. 1A RA-T, FIG. 7 and FIG. 8), a third conduction-blocking lesion extending laterally along the right atrium and intersecting the second conduction-blocking lesion (See, e.g., FIG. 1A RA-LATERAL, FIG. 9 and FIG. 11), a fourth conduction-blocking lesion in the coronary sinus (See, e.g., FIG. 1A Coronary Sinus, FIG. 12 and FIG. 15), a fifth conduction-blocking lesion extending along a transverse line located below the right and left inferior pulmonary veins (See, e.g., FIG. 1A Inferior LPV, FIG. 16, and FIG. 20), a sixth conduction-blocking lesion extending along a transverse line located above the right and left superior pulmonary veins (See, e.g., FIG. 1A Superior LPV, FIG. 21 and FIG. 23), a seventh conduction-blocking lesion comprised of a plurality of lesions extending along the anterior interatrial groove proximate the origins of the right superior and inferior pulmonary veins and intersecting the fifth conduction-blocking lesion below the right inferior pulmonary vein and the sixth conduction-blocking lesion above the right superior pulmonary vein (See, e.g., FIG. 1A R PV and FIG. 25B) and an eighth conduction-blocking lesion located along a line extending from the base of the left atrial appendage to a location proximate the mitral annulus (See, e.g., FIG. 1A LAA, FIG. 26 and FIG. 28). Although the lesions are labeled first, second, third, etc., this is for ease of reference and the lesions may be made in any order.

The interventional procedures described herein may be accomplished in a closed-chest procedure using a minimally invasive access technique, such as, small left mini-thorocotomy, scope, and the like. The interventional procedures described herein may be performed using any surgical or electrophysiological technique or any combination therefore. In some embodiments, the interventional procedures described herein may be performed utilizing an interdisciplinary approach referred to as a “Hybrid Procedure.” As used herein, the term “Hybrid Procedure” refers to an interventional procedure that employs both surgical and electrophysiological techniques. In some embodiments, an electrophysiologist performs one or more lesions while a surgeon watches through a subxiphoid scope to observe the location and completeness of the lesions being created. In some embodiments, an electrophysiologist performs the right atrial (RA) lesions while a surgeon watches through a subxiphoid scope to observe the location and completeness of the lesions being created. In some embodiments, a surgeon performs the left atrial (LA) lesions while an electrophysiologist watches through an endocardial scope to observe the location and completeness of the lesions being formed. The interventional procedures described herein may also be performed by one or more physicians of any discipline that performs cardiac procedures.

Several embodiments described herein relate to a hybrid interventional procedure that adheres to the principle of a maze of lesions to isolate and direct cardiac rhythm signals and does not require the use of a heart-lung machine. In some embodiments, right atrial lesions may be performed by electrophysiological techniques. In some embodiments, right atrial lesions may be verified as being complete by direct observation of the epicardium. In some embodiments, minimally invasive access techniques, such as the use of a subxiphoid endoscope and/or a small left atriotomy, may be employed. In some embodiments, access into the left atrial cavity may be gained through the left atrial appendage. In some embodiments, access into the left atrial cavity may be gained through the left atrial appendage. In some embodiments, a lesion located along a line extending from the base of the left atrial appendage to a location proximate the mitral annulus (sub-LAA lesion) may be made as an alternative to the mitral line and coronary sinus lesions used in previous Maze procedures. In some embodiments, a lesion extending from the tip of the right atrial appendage (RAA) to the “T” lesion on the lateral right atrium (RA Lateral Lesion) may be made as an alternative to the “counterlesion” used in previous Maze procedures.

Conduction blocking lesions may be formed using any method or device that traumatically damages cardiac tissue resulting in the formation of conduction blocking scar tissue. For example, lesions may be formed by surgical cutting or by application of ablative energy, such as cryogenic, high intensity focused ultrasound (HIFU), laser energy, radiofrequency (RF) energy, heat energy and/or microwave energy. In some embodiments, lesions are formed by applying ablative energy to the epicardium. In other embodiments lesions are formed by applying ablative energy to the endocardium. In some embodiments, ablative energy may be applied to both the endocardial and epicardial surfaces, either simultaneously or sequentially. Ablative energy may be applied to epicardial and/or endocardial surfaces using surgical, intravascular and/or other minimally invasive techniques, such as percutaneous, small incisions or ports. The application of ablation energy (e.g., phase, magnitude, pulse sequence, etc.), type of ablation energy (e.g., radiofrequency, laser, high intensity focused ultrasound, cryogenic agents, microwave energy, heat energy, etc.), as well as the positioning and the shape and size of the ablation device may be varied according to the geometry of the tissue and the ablation profile desired. For example, in some embodiments, one or more lesions may be formed by cryogenic endocardial ablation, while one or more other lesions may be formed by epicardial application of heat energy. Alternatively, in some embodiments, one or more lesions may be formed by the endocardial application of heat energy, while one or more other lesions may be formed by cryogenic epicardial ablation. In other variations, one or more lesions may be formed by the endocardial application of HIFU, while one or more other lesions may be formed by the epicardial application of microwave energy. The type(s) of ablation energy used as well as the positioning, type and the shape and size of the ablation device may be selected to limit damage to non-target peripheral tissue.

The ablation device may be a probe, a pair of probes, a clamp, a catheter, a balloon-catheter, as well as any other device deliverable or otherwise positionable proximate to a tissue region for treatment through the vasculature and/or by gaining access to the pericardial space. In some embodiments, the ablation device or a pair of ablation devices may be configured to apply ablative energy to both the endocardial and epicardial surfaces to ablate the cardiac tissue from both sides. Application of ablative energy simultaneously from both sides may help promote the formation of a lesion that spans a significant portion of the thickness of the tissue. Some ablation devices may ablate tissue using a combination of different mechanisms, as suitable for the target tissue. In some embodiments, ablation device may include one or more sensors to monitor the operating parameters throughout the system, including for example, pressure, temperature, flow rates, volume, or the like.

The interventional procedures described herein comprise a more complete set of conduction-blocking lesions than other minimally invasive epicardial surgical procedures or endocardial electrophysiology procedures, such as the Mini-Maze and Left-sided Maze procedures; yet Maze-VII procedures retain their advantages of being minimally invasive and not requiring cardiac arrest and use of a heart-lung machine. The embodiments described herein overcome the tradeoff between the lack of efficacy of catheter ablation for AF treatment and the excessive invasiveness of traditional surgical treatments for AF.

The present embodiments further overcome the limitations imposed by the instruments available for the treatment of AF by either interventional method. For example, in treatment methods where ablative energy is applied to the epicardium “off-pump” (not using a heart-lung bypass pump), the intracavitary blood pool acts as a heat sink for cryosurgical devices and as a cooling sink for heat-based energies such as RF, HIFU, microwave, and laser, making it difficult to create reliable transmural lesions, since there is no way to determine whether the ablation lesion is fully transmural or not. This is problematic because if the lesions are not contiguous and transmural, they will not cure AF, even if they are placed in a correct maze pattern. While the cooling sink effect of the cavitary blood can be overcome by applying the heat-based energy sources from the endocardium, there is no visual way to determine if a lesion is contiguous and transmural. Non-visual sensing methods are more complex and less reliable than simple visualization. For example, the most common solution to the problem of determining whether a lesion is sufficiently contiguous and transmural to form a conduction block is to perform immediate electrophysiologic testing of the integrity of lesions after applying them with either a catheter or a surgical device; however this immediate post-procedure electrophysiologic testing is unreliable and of limited value because the target tissue may be damaged enough to create a temporary conduction block, but not damaged enough to create a permanent conduction block.

In several embodiments described herein, the heat-sink or cooling-sink problems due to atrial cavitary blood with off-pump epicardial ablation are overcome by applying the ablative energy from the endocardium and observing the resultant tissue damage from the epicardium. In some embodiments, cryogenic ablation energy is applied through the endocardium until a cryogenic “iceball” penetrating the epicardium is observed. This technique allows for real time visual confirmation that a given lesion is transmural throughout its length and provides an extremely effective way of creating transmural lesions in “off-pump” procedures where cavitary blood creates an energy sink.

Several embodiments described herein relate to systems, methods, and medical devices for providing a maze pattern of conduction-blocking lesions in the heart optimized for use with minimally invasive interventional surgical and electrophysiological techniques to treat all forms of AF.

Referring to FIGS. 12-15, a lesion may be placed inside the coronary sinus. In some embodiments, the coronary sinus lesion may be placed using a catheter comprised to include an ablation energy surface at its distal end. Catheter access may be through the vena cava or other such suitable route amenable to catheter navigation. The ablation energy source may be any of those described herein. In some embodiments, ablation energy source is a cryogen. The ablation energy source may be further comprised to include an expanding member that may be expanded to contact the inner lumen of the coronary sinus. The expanding member may be in any form sufficient to contact and conform to the shape of the coronary sinus inner lumen. In some embodiments, the expanding member may be an inflatable balloon configured to transmit ablative energy for the creation of a lesion. In some embodiments, the expanding member may be an expandable framework, such as a basket, configured to transmit the ablative energy. The ablation energy source may be of any length suitable for sufficient energy transfer. For example, the ablation energy source may be of a length that minimizes the number of ablation cycles necessary to form a lesion of sufficient surface area to block macro-reentrant circuits. Optionally, the formation of the lesion may be observed endoscopically, for example by using a scope placed through a subxiphoid access incision.

In an alternate embodiment for forming the coronary sinus lesion, a probe comprising an ablation energy source may be used to create the lesion. In some embodiments, the probe may create the lesion by being placed through an access point in the heart. For example, the probe may be placed through an access point in the right atrial appendage, optionally using means such as a purse string suture or valved sheath to prevent the escape of blood from the beating heart. In some embodiments, the probe may be configured to comprise an expanding structure such as a balloon, a basket, a coil, or the like, as part of the means for delivering ablation energy, for example, cryogenic energy to the targeted tissue.

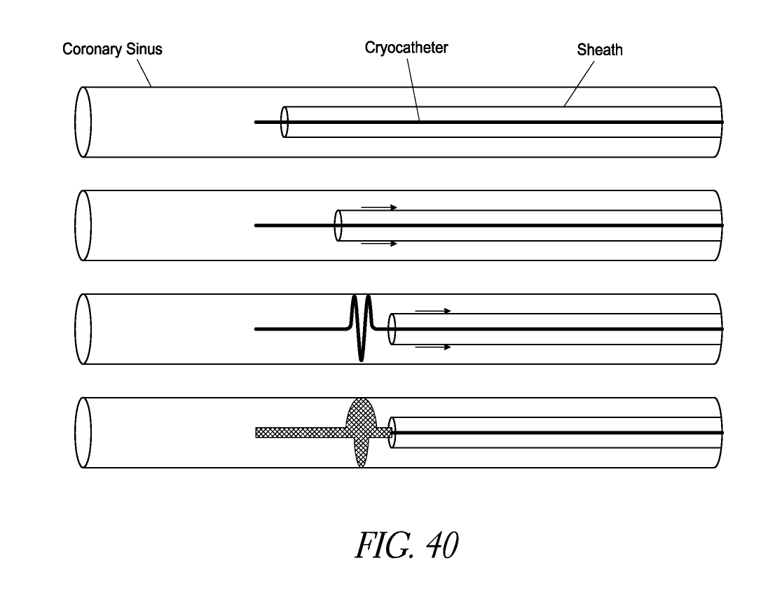

Referring again to FIGS. 12-15, a circumferential lesion in the coronary sinus may be created. In some embodiments, a circumferential lesion in the coronary sinus may be created using a catheter that is introduced inside a sheath. In some embodiments, the catheter may be configured to spring open when the constraints of its external insertion sheath are removed. In some embodiments, when the sheath is withdrawn far enough to allow the expandable structure in the catheter to “spring open,” the catheter may engage the coronary sinus circumferentially. In some embodiments, the catheter is a cryocatheter through which cryogen may then be circulated to circumferentially ablate the coronary sinus. Referring to FIG. 40, in some embodiments, the distal portion of the catheter may be configured to comprise a coil or loop-like feature or other features that are unconstrained and which can be formed by mechanical action or by thermal action in embodiments where a shape memory alloy is used. In an embodiment where the catheter is configured to comprise a single loop-like feature, the feature provides locating force against the endocardium and also provides the working ablative surface for lesion formation. In an embodiment where the catheter is configured to comprise a plurality of loop-like features, one or more features may be used for locating and securement, while one or more features may be used for ablation. Hooks, barbs or other such securement means may be further used to aid in securement in any endocardial probe embodiment. The tissue contacting portion of the catheter may be comprised of any suitable biocompatible material or combination of materials, such as, metals such as stainless steel or Nitinol, and plastics, such as mylar, Kapton or polyamide.

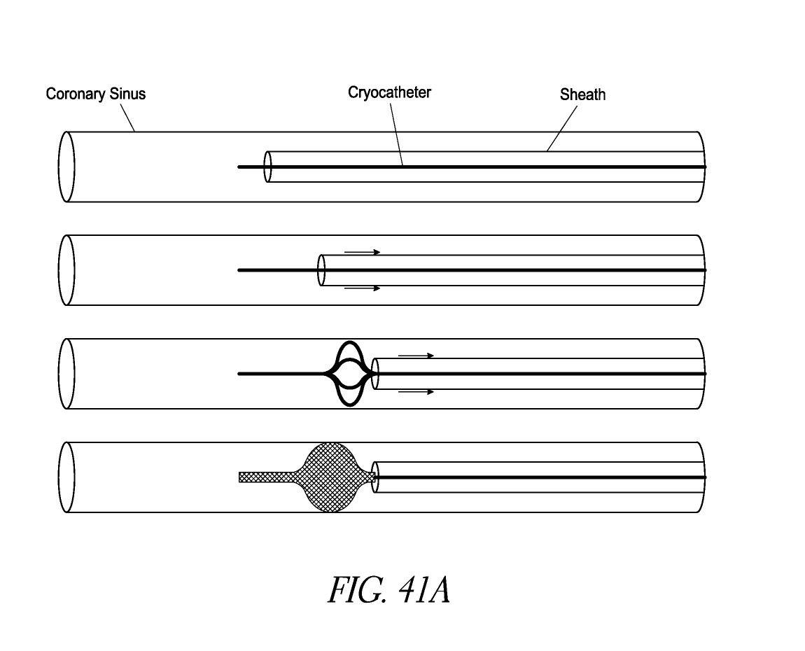

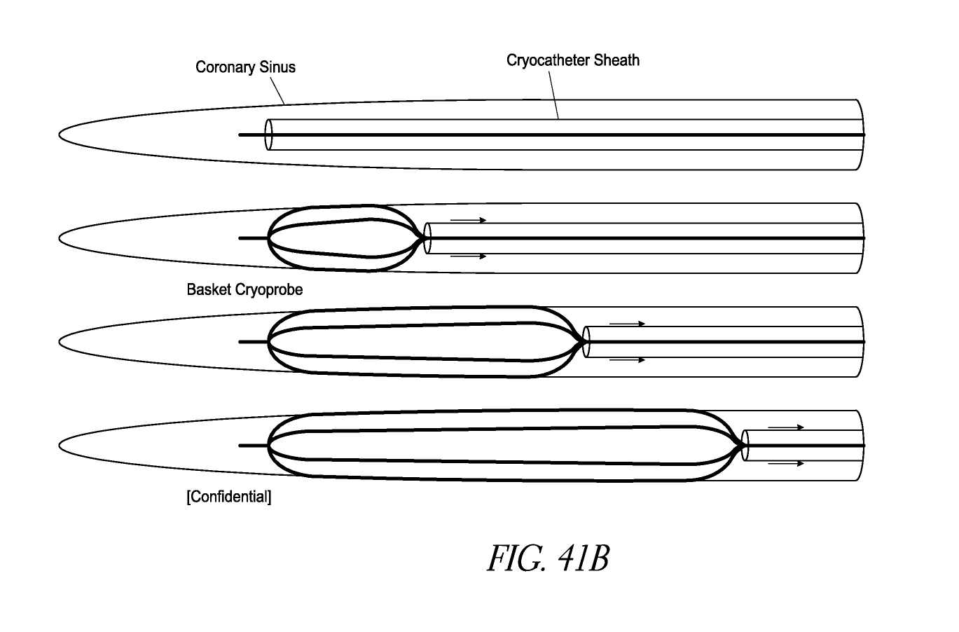

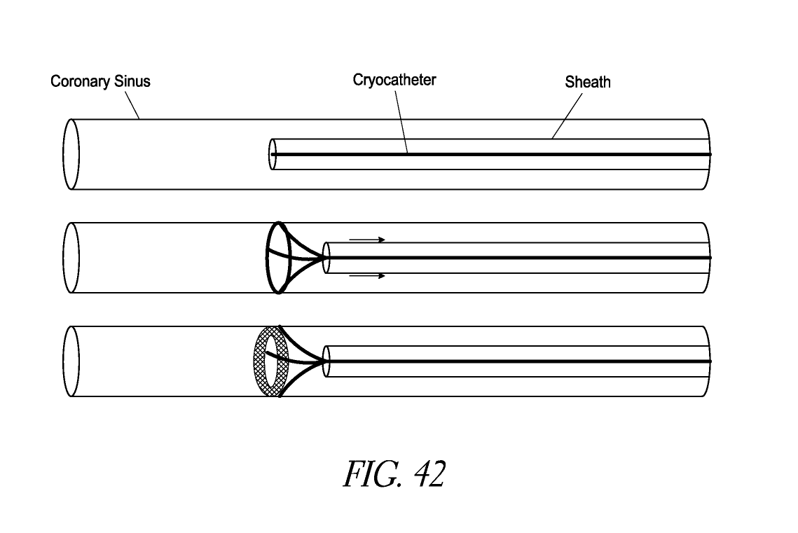

Referring now to FIGS. 41A and 41B, the ablation surface comprising a distal portion of the catheter may be comprised of a structure that may spring open to form a basket shape when the catheter sheath is retracted such that it may provide a means for creating a circumferential lesion around the coronary sinus. The basket structure may provide a framework upon which one or more ablation members are mounted, or alternately, the basket structure itself may comprise ablation member such as lumens for cryogens, RF electrodes, HIFU transducers, and the like. Referring to FIG. 42, the ablation surface comprising a distal portion of the catheter may be comprised of a structure that may spring open to form a flange or bell-like shape when the catheter sheath is retracted.

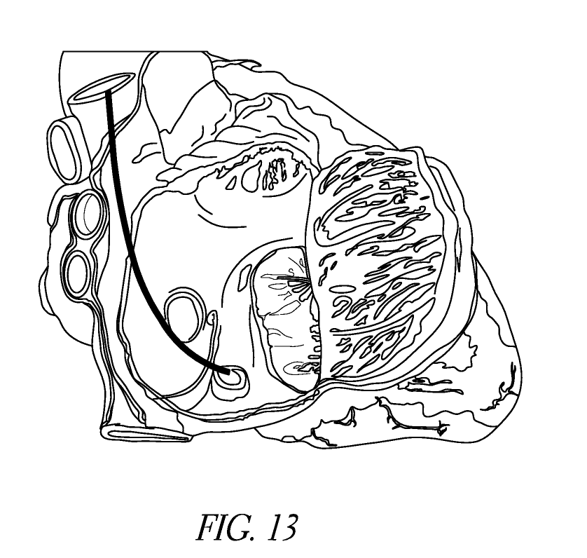

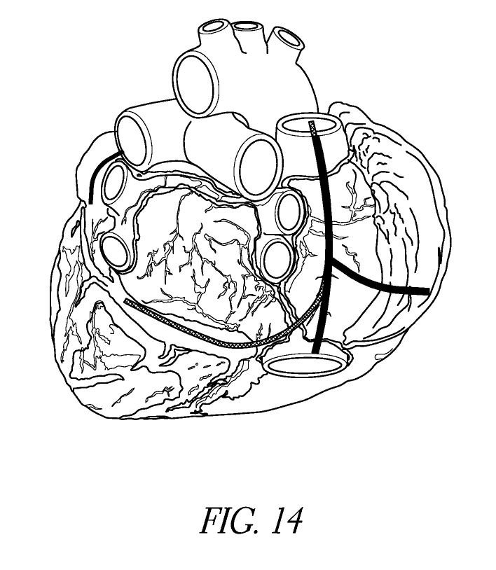

As shown in FIGS. 13 and 14, the ablation device, for example, a cryocatheter and sheath, is passed into the ostium of the coronary sinus in the right atrium. The ablation device tip is then passed retrograde into the coronary sinus as far to the left side as possible. As the sheath is then withdrawn, the structure of the internal ablation member is expanded. The surface area of the coronary sinus is ablated along a length of about 15 cm, as shown schematically in FIGS. 12 and 15. The working length of the ablation member may be of any length to allow for an instrument with good navigability and access size. In some embodiments, the ablation member may be of a length to allow for one, two or three ablative steps to form a completed lesion.

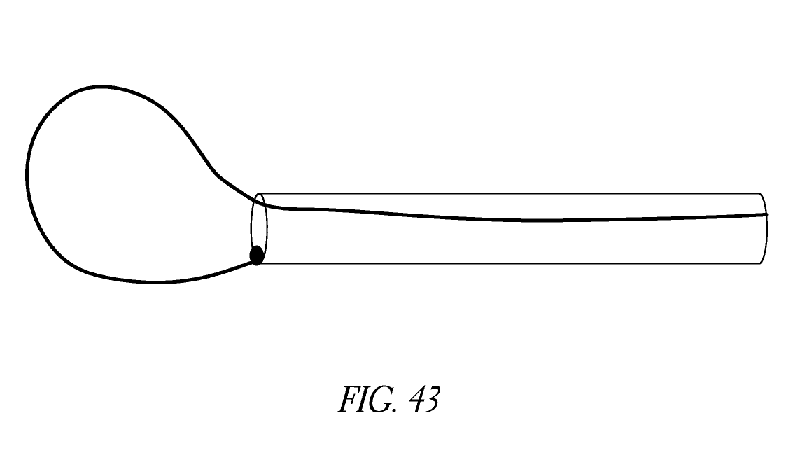

Referring now to FIG. 43, the distal end of the catheter may be comprised of an inner member that may be actuated to form a loop by pushing the member out of the sheath. An end of the inner member is fixed within the inner lumen of the sheath near its distal end such that a bow or loop-like shape is formed as an increasing amount of the inner member is pushed out of the sheath tip. In some embodiments, the bow may be large enough to ablate both the coronary sinus and for completing the final lesion to isolate the PV's as shown in FIG. 25B.

Optional for all cryogenic ablation embodiments described herein, the ablative surface may comprise a means of producing enough heat at the end of the freeze to quickly disconnect the ablative surface from the cryolesion itself by cessation of cooling, by a warming cycle, by thawing, or the like.

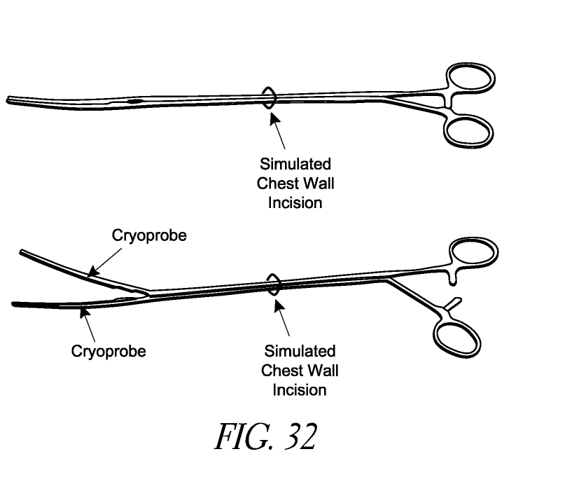

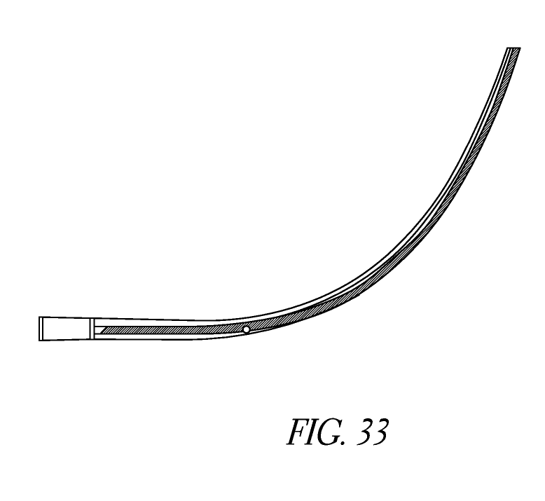

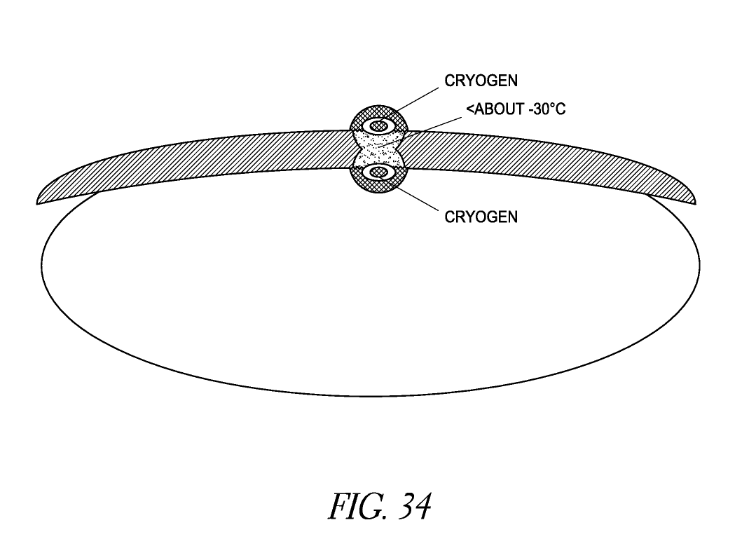

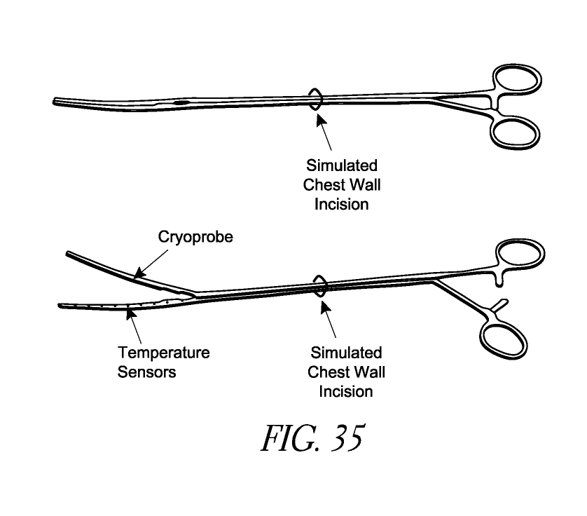

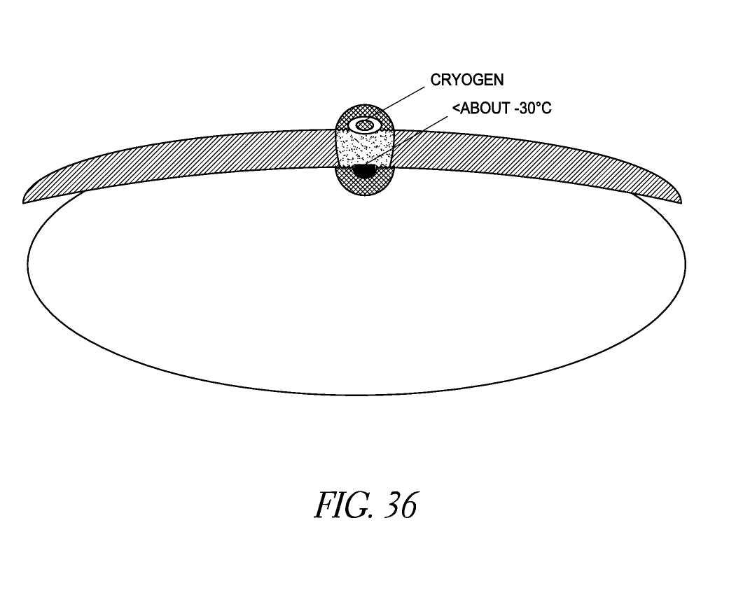

Referring to FIGS. 32-36, a clamp comprising an ablation member may be used to create ablation lesions. The clamp is configured to have two opposing jaws that when actuated may open or close to apply pressure there between. One jaw of the clamp may be placed along the surface of the endocardium through a further access point through the heart, the other jaw of the clamp may remain external to the heart along the surface of the epicardium such that the wall of the heart is positioned between the jaws of the clamp and subjected to pressure when the clamp jaws are actuated closed. As shown in FIGS. 32 and 34, both clamp jaws are comprised of a “bipolar” ablation means such that a transmural lesion may be made from both the internal and external surfaces of the heart adjacent the clamp. As shown in FIGS. 35 and 36, one jaw is comprised of an ablation means and the other jaw is comprised of a temperature sensing means configured to detect a temperature indicative of a lesion that has reached a sufficient state of transmurality. In some embodiments the jaws of the clamp may be a configured to allow for actuation independent of one another. The clamp may optionally be configured to steer or be bent so that the right-side T lesion (FIG. 7) may be made at about 90 degrees from the point of access or as much as about 180 degrees to form the right lateral lesion (FIG. 9). In some embodiments, the clamp may be constructed of a flexible material that may allow the clamp to be bent to the preferred shape and then inserted and positioned across the right side of the heart to make the desired lesion. Optionally, the inside of both jaws may be recessed to provide a groove into which the cryogenic lumen and/or temperature sensors are situated.

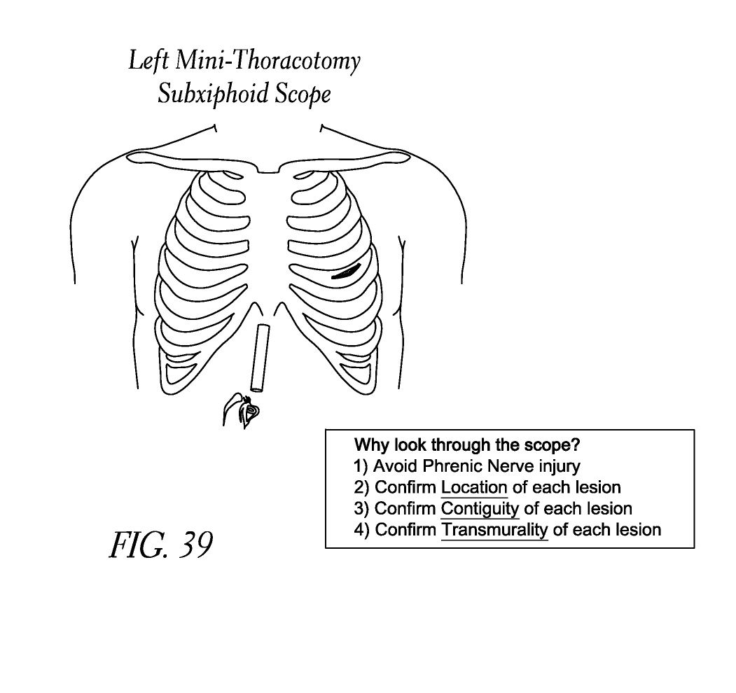

In some embodiments, the cryosurgical clamp may have a thin shaft so that it may be introduced though a very small opening, such as that of a mini-thorocotomy in the chest wall or an endoscope, such access points being schematically shown in FIG. 39. For example, when closed, the clamp may be inserted through a very small chest wall incision. After it is positioned inside the chest, the clamp jaws may be opened wide enough to preferably be able to clamp large structures; the clamp may be manipulated by the clamp's handle which is well outside the chest.

In embodiments using Super-Critical Nitrogen (SCN) cryogen, the cryogenic lumen may be miniaturized to provide a particularly small access profile. SCN may provide for probe temperatures as low as about −195° C. while having the heat capacity of a liquid rather than a gas. Other cryogens currently being used clinically are Nitrous Oxide gas, which cools the probe down to about −60° C.; and Argon gas, which cools the probe down to about −160° C. As shown in FIG. 34, the cryogen may be applied from both the endocardium and the epicardium until the middle of the atrial wall reached the “nadir” (uniformly fatal) temperature of −30° C. In thin atrial walls, ablation time may be as low as a few seconds. Alternately, as shown by FIG. 36, the cryogen may be applied from one side of the heart, most preferably the epicardium but also from the endocardium (not shown).

Referring again to FIG. 35, a unipolar cryosurgical clamp may be comprised to have a cryoprobe on one jaw and a plurality of thermistors on the other jaw. Having a clamp with thermistors on the jaw opposite the cryogenic lumen, the cryogen could be placed from either the epicardium or the endocardium. As shown in FIG. 36, the cryogen is being applied epicardially with a unipolar cryosurgical clamp while the endocardial temperature is being monitored by the plurality of thermistors. A transmural lesion may be indicated by a plurality of thermistors indicating a temperature of about −30° C. or lower. In an alternate embodiment, the cryosurgical clamp may be unipolar, having no thermistors on the jaw opposite the cryogenic lumen.

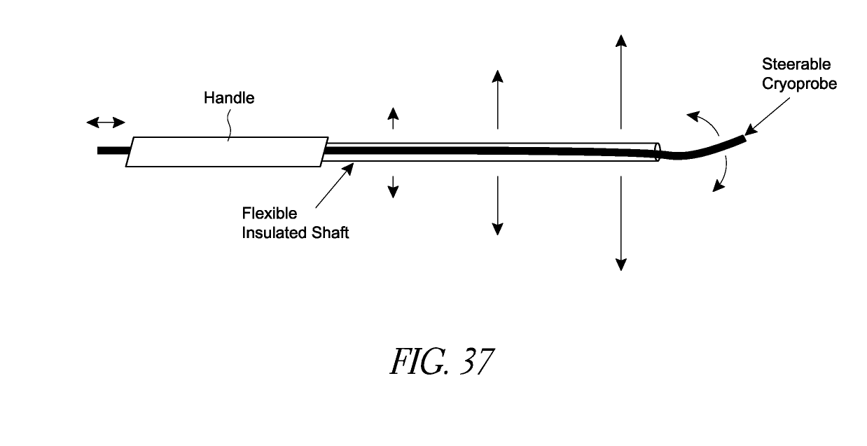

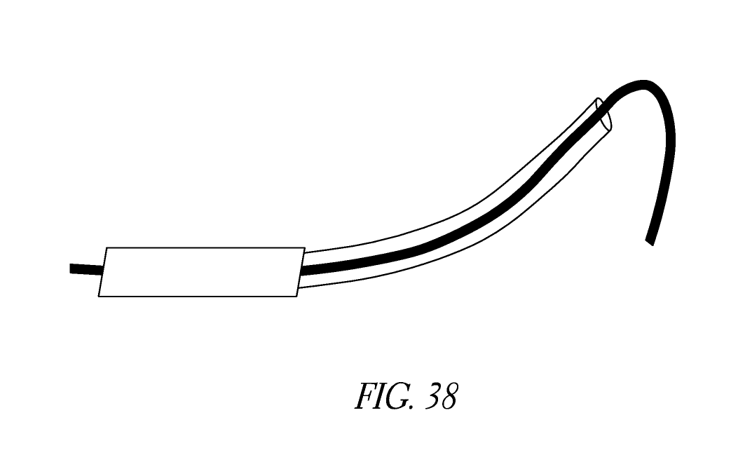

Referring now to FIGS. 37 and 38, a steerable cryoprobe may be used to perform some of the lesions of the procedure described herein. Probes may be comprised of an ablation surface at its distal portion with a preferred diameter of about 3 mm to provide a desirable combination of access size, stiffness, and working surface area, however, the distal end may be of any size and shape sufficient to form the lesions described herein. The curvature of approximately the distal two inches of the cryoprobe may be controllable from the handle and be capable of one or both shaft curvature and tip curvature as depicted in FIGS. 37 and 38. The inner cryoprobe may preferably be freely moveable through the handle and shaft so that it can be lengthened or withdrawn completely inside the shaft.

In some embodiments, the shaft of the instrument may provide sufficient stiffness to provide strength when pressure is applied for surface contact during lesion formation while remaining malleable so that it can be shaped. In one embodiment, steering or shaping may be performed by hand before insertion and use. In some embodiments, the overall size of the instrument is about 10-12 inches long with a cryoprobe of about 3 mm diameter. In some embodiments, the probe comprises a slightly larger diameter shaft and a slightly larger diameter handle. FIG. 37 shows the cryoprobe instrument with the shaft straight and the probe curved almost 180 degrees. FIG. 38 shows the cryoprobe instrument with the shaft bent upward and the probe itself deflected in the opposite direction.

Right Atrium:

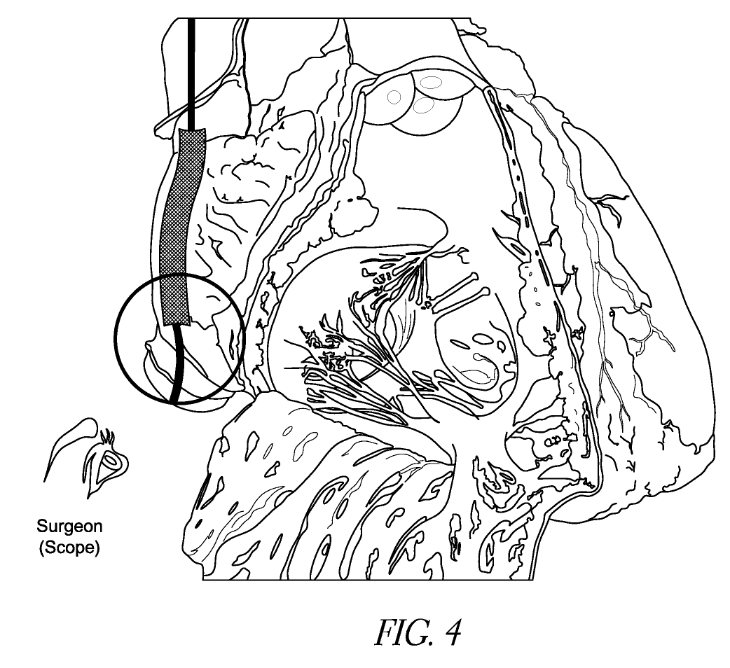

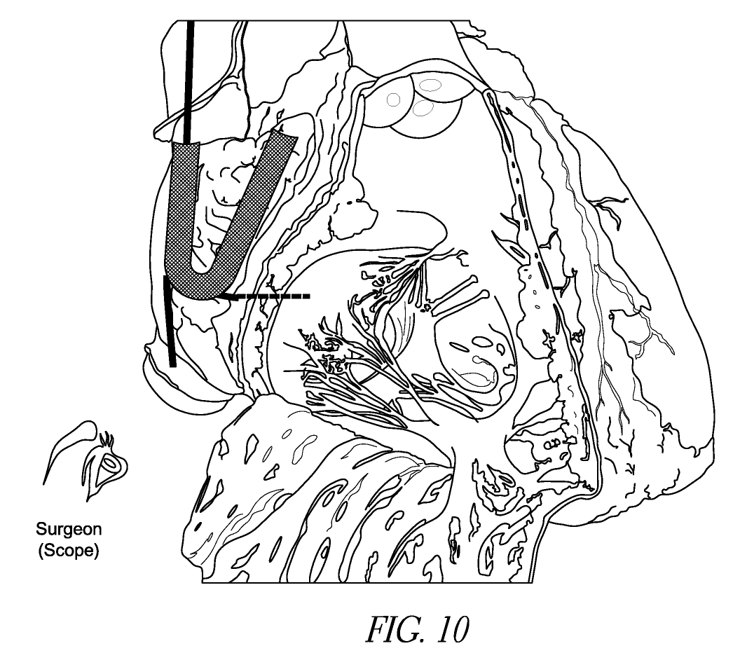

Referring to FIGS. 3-11, the “counterlesion” that was not present in the Maze-I but was added to all subsequent iterations of the Maze procedure to prevent a potential macro-reentrant circuit around the base of the right atrial appendage; this lesion may be deleted if the old “right atrial lateral lesion” (FIGS. 9-11) is simply continued from the tip of the RAA down to the “T” lesion (FIGS. 7 and 8) on the lateral RA.

The RA lesions may be performed by the interventional EP as a surgeon observes the lesion formation via the subxiphoid scope as shown in FIG. 39. In some embodiments, the scope is inserted into the pericardium through fluid-tight opening in the pericardium such that the pericardium may be distended away from the heart with insufflation using means such as CO₂, saline, or a more viscous solution comprised of a biocompatible substance having a viscosity not less than that of saline. In embodiments using insufflations, the pericardium may preferably be held away from the heart so as to provide an improved view of the surface of the heart as compared to the view of the heart typically seen through a scope.

Referring now to FIGS. 3-6, in one embodiment, the first lesion to be performed may be between the Superior Vena Cava (SVC) and the Inferior Vena Cava (IVC). In the embodiment shown by FIG. 3, a lesion along the superior to inferior vena cava may be made using a catheter comprised to include an ablation member at or near its distal end. Ablation energy may be of any of the sources described herein. In some embodiments, the ablation energy source is a cryogen. In some embodiments, the catheter delivers ablation energy from the endocardium transmurally to the epicardium. Optionally, the formation of the lesion may be observed endoscopically using a means such as a scope placed through a subxiphoid access incision.

Further optionally, a probe may be placed through a lumen in the scope such that it may be used as a means for one or more of ablation guidance, application of pressure between the working portion of the ablation member, temperature monitoring, and protection of tissues adjacent to the lesion.

In some embodiments, for producing the superior to inferior vena cava lesion, a probe comprising an ablation member may be used to create the lesion. In some embodiments, the probe may create the transmural lesion from the epicardium and may be passed through a lumen in the scope or may be passed through a secondary access port in the thorax or abdomen. In some embodiments, the probe may create the transmural lesion from the endocardium by being placed through a further access point through the heart. In some embodiments, access is gained through the right atrial appendage using means such as a purse string suture or valved sheath that may prevent the escape of blood from the beating heart.

In some embodiments for producing the superior to inferior vena cava lesion, a clamp comprising an ablation member may be used to create the lesion and may be passed through a secondary access port in the thorax. The clamp is configured to have two opposing jaws that when actuated may open or close to apply pressure there between. One jaw of the clamp may be placed along the surface of the endocardium through a further access point through the heart. In some embodiments, access is gained through the right atrial appendage using means such as a purse string suture that may prevent the escape of blood from the beating heart. The other jaw of the clamp may remain external to the heart along the surface of the epicardium such that the wall of the heart is positioned between the jaws of the clamp and subjected to pressure when the clamp jaws are actuated closed. In one embodiment, both clamp jaws are comprised of an ablation member configured such that a transmural lesion may be made from both the internal and external surfaces of the heart adjacent the clamp. In another embodiment, one jaw is comprised of an ablation member and the other jaw is comprised of a temperature sensor configured to detect a temperature indicative of a lesion that has reached a sufficient state of transmurality. In some embodiments the jaws of the clamp may be a configured to allow for actuation independent of one another.

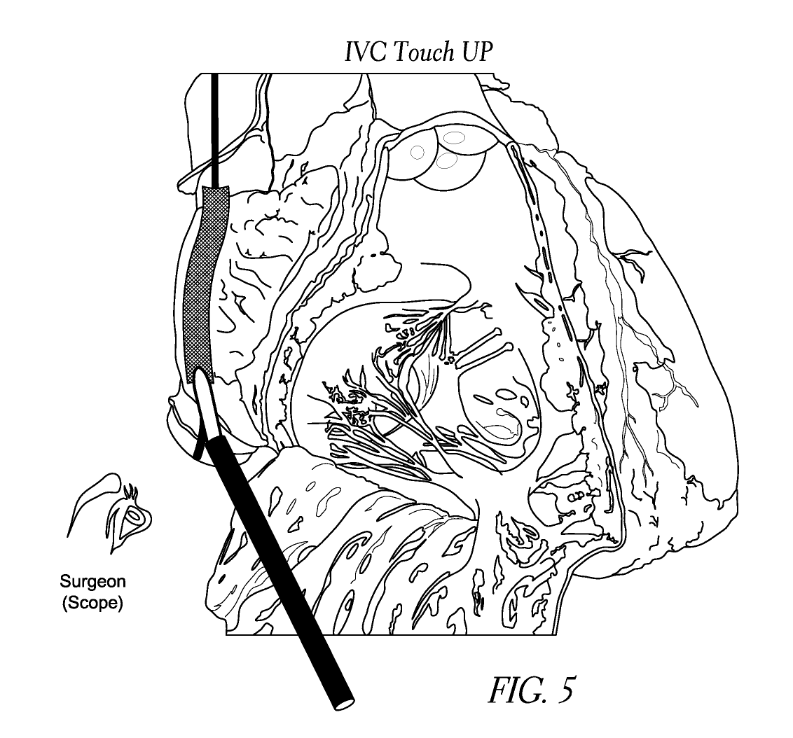

As shown in FIG. 5, the surgeon has optionally passed a cryoprobe through the scope to “touch up” the portion of the endocardial lesion that was incomplete, thus preventing a failure of the procedure due to escape of macro re-entrant signals. It is important to note that the right Phrenic Nerve runs extremely close to this lesion at the level of the pericardial reflection off the IVC. Thus, in some embodiments, damage to the Phrenic Nerve may be avoided by the “protection” of the surgeon being able to see where this lesion is being placed. In some embodiments, the probe may create the transmural lesion from the epicardium and may be passed through a lumen in the scope or may be passed through a secondary access port in the thorax or abdomen. Additionally, the probe may further comprise an insulation sheath or other such similar adjustable means by which to control the amount of surface area on the working portion of the ablation member such that a more precise control of ablation lesion formation may be achieved in areas where sensitive tissue may be adjacent to the targeted lesion zone. By way of example, if an insulating sheath were actuated to expose only the very most tip of the ablation member, the fine tuning of the lesion may be accomplished with increased precision in a manner analogous to the drawing of a line on paper using a marking pen and a fine-tipped pen.

Referring now to FIGS. 7 and 8, in one embodiment, the second lesion may be created by the electrophysiologist and is referred to as the “T” lesion across the lower right atrial free-wall. In some embodiments, an endocardial catheter may be placed in a curved manner against the lateral free-wall of the right atrium so that a lesion may be placed from the SVC-IVC lesion to the tricuspid annulus. Ablative energy may then be applied to complete the T lesion. By extending this lesion from the tip of the RA appendage to the “T” lesion, in some embodiments, placement of the “counterlesion” from the tip of the RAA to the tricuspid annulus may be forgone. The right-side “T” lesion is roughly perpendicular to the superior to inferior vena cava and in some embodiments may be created using the same or similar variety of means used to create the superior to inferior vena cava lesion.

In one embodiment for creating the right-side T lesion, an endocardial catheter, comprised to include an ablation member at or near its distal end, may be used to conduct ablation energy of any of the sources described herein. In some embodiments, the ablation energy source conducted by the ablation member is a cryogen. In some embodiments, a catheter delivers ablation energy from the endocardium transmurally to the epicardium. The catheter may be configured to steer or otherwise be turned about 90 degrees to the direction to the axis of the vena cava so that it may be positioned to create a lesion across the right side of the heart transverse to the vena cava, in some embodiments, about mid way between the superior and inferior vena cava and traversing the right side of the heart. Optionally, the formation of the lesion may be observed endoscopically using a means such as a scope placed through a subxiphoid access incision.

In some embodiments for producing the right-side T lesion, a probe comprising an ablation member may be used to create the lesion. In some embodiments, the probe may create the transmural lesion from the epicardium and may be passed through a lumen in the scope or may be passed through a secondary access port in the thorax or abdomen. In some embodiments, the probe may create the transmural lesion from the endocardium by being placed through a further access point through the heart. In some embodiments, access is gained through the right atrial appendage using means such as a purse string suture or valved sheath that may prevent the escape of blood from the beating heart. The probe may be configured to steer or be bent so that the roughly 90 degree turn from the vena cava may be accomplished. In some embodiments, the probe may be constructed of a flexible material that may allow the probe to be bent to the preferred shape and then inserted into the vena cava to navigate transverse from the vena cava across the right side of the heart to make the desired lesion.

In some embodiments for producing the right-side T lesion, a clamp comprising an ablation member may be used to create the lesion and may be passed through a secondary access port in the thorax. The clamp is configured to have two opposing jaws that when actuated may open or close to apply pressure there between. One jaw of the clamp may be placed along the surface of the endocardium through a further access point through the heart. In some embodiments, access may be gained through the right atrial appendage using means such as a purse string suture that may prevent the escape of blood from the beating heart. The other jaw of the clamp may remain external to the heart along the surface of the epicardium such that the wall of the heart is positioned between the jaws of the clamp and subjected to pressure when the clamp jaws are actuated closed. In one embodiment, both clap jaws are configured to comprise an ablation member configured such that a transmural lesion may be made from both the internal and external surfaces of the heart adjacent the clamp. In another embodiment one jaw is comprised of an ablation member and the other jaw is comprised of a temperature sensor configured to detect a temperature indicative of a lesion that has reached a sufficient state of transmurality. In some embodiments, the jaws of the clamp may be a configured to allow for actuation independent of one another. The clamp may optionally be configured to steer or be bent so that the right-side T lesion may be made at about 90 degrees from the point of access. In some embodiments, the clamp may be constructed of a flexible material that may allow the clamp to be bent to the desired shape and then inserted and positioned across the right side of the heart to make the desired lesion.

Referring now to FIGS. 9-11, the lateral RA lesion may be placed in some embodiments by curving the endocardial cryocatheter so that it extends from the “T” lesion up to the tip of the RAA, and then applying ablative energy to complete the RA lesions. In some embodiments, the RA lesions may be performed by an electrophysiologist. In some embodiments, the RA lesions may be performed by a cardiologist.

In one embodiment for creating the right-side lateral lesion, an endocardial catheter, comprised to include an ablation member at or near its distal end, may be used to conduct ablation energy of any of the sources described herein. In some embodiments, the energy source conducted by the ablation member is a cryogen. In some embodiments, the catheter delivers ablation energy from the endocardium transmurally to the epicardium. The catheter may be configured to steer or otherwise be turned about 180 degrees to the direction to the axis of the vena cava so that it may be positioned to create a lesion extending roughly perpendicular from the right-side T and terminating in proximity to the right atrial appendage. Optionally, the formation of the lesion may be observed endoscopically using a means such as a scope placed through a subxiphoid access incision.

In one embodiment for producing the right-side lateral lesion, a probe comprising an ablation member may be used to create the lesion. In some embodiments, the probe may create the transmural lesion from the epicardium and may be passed through a lumen in the scope or may be passed through a secondary access port in the thorax or abdomen. In some embodiments, the probe may create the transmural lesion from the endocardium by being placed through a further access point through the heart. In some embodiments, access is gained through the right atrial appendage using means such as a purse string suture or valved sheath that may prevent the escape of blood from the beating heart. The probe may be configured to steer or be bent so that the roughly 180 degree turn from the vena cava may be accomplished. In some embodiments, the probe may be constructed of a flexible material that may allow the probe to be bent to the preferred shape and then inserted into the vena cava to navigate transverse from the vena cava across the right side of the heart and about 180 degrees to make the desired lesion vertically along the right atrium.

In one embodiment for producing the right-side lateral lesion, a clamp comprising an ablation member may be used to create the lesion and may be passed through a secondary access port in the thorax. The clamp is configured to have two opposing jaws that when actuated may open or close to apply pressure there between. One jaw of the clamp may be placed along the surface of the endocardium through a further access point through the heart. In some embodiments, access is gained through the right atrial appendage using means such as a purse string suture that may prevent the escape of blood from the beating heart. The other jaw of the clamp may remain external to the heart along the surface of the epicardium such that the wall of the heart is positioned between the jaws of the clamp and subjected to pressure when the clamp jaws are actuated closed. In one embodiment, both clap jaws are configured to comprise an ablation member configured such that a transmural lesion may be made from both the internal and external surfaces of the heart adjacent the clamp. In another embodiment, one jaw comprises an ablation member and the other jaw comprises a temperature sensor configured to detect a temperature indicative of a lesion that has reached a sufficient state of transmurality. In some embodiments, the jaws of the clamp may be a configured to allow for actuation independent of one another. The clamp may optionally be configured to steer or be bent so that the right-side lateral lesion may be made at about 180 degrees from the point of access. In some embodiments, the clamp may be constructed of a flexible material that may allow the clamp to be bent to the preferred shape and then inserted and positioned across the right side of the heart to make the desired lesion.

Coronary Sinus:

Referring now to FIGS. 12-15, in one embodiment, a fourth lesion may be placed in the coronary sinus from a right-side access through the ostium of the coronary sinus in the right atrium as shown in FIG. 13, with the ablation tool being passed retrograde into the coronary sinus as far to the left side as possible.

In some embodiments, the coronary sinus lesion is made by a catheter comprised to include an ablation member at its distal end. Catheter access may be through the vena cava or other such suitable route amenable to catheter navigation. The ablation energy source may be any of those described herein. In some embodiments, the ablation energy source is a cryogen. The ablation member may be further comprised to include an expanding member that may be expanded to contact the inner lumen of the coronary sinus. The expanding member may be in any form sufficient to contact and conform to the shape of the coronary sinus inner lumen. In one embodiment, the expanding member may be an inflatable balloon configured to transmit the ablative energy for the creation of a lesion. In some embodiments, the expanding member may be an expandable framework, such as a basket, configured to transmit the ablative energy. The ablation member may be of any length suitable for sufficient heat transfer. In some embodiments, the ablation member is of a length that minimizes the number of ablation cycles necessary to form a lesion of sufficient surface area to block macro-reentrant circuits. Optionally, the formation of the lesion may be observed endoscopically using a means such as a scope placed through a subxiphoid access incision.

In one embodiment for forming the coronary sinus lesion, a probe comprising an ablation member may be used to create the lesion. In some embodiments, the probe may create the lesion by being placed through an access point in the heart. In some embodiments, access is gained through the right atrial appendage using means such as a purse string suture or valved sheath that may prevent the escape of blood from the beating heart.

Any of the embodiments of probe or catheter may be configured to comprise an expanding structure such as a balloon, a basket, a coil, or the like, as part of the means for delivering ablation energy of the types described herein.

Left Atrium:

In one embodiment, the LA lesions may be performed by a surgeon via the left mini-thorocotomy. In one embodiment as shown by FIG. 39, a mini-thorocotomy of any size sufficient for the purpose, for example a mini-thorocotomy of about 4-5 cm length, may be performed in or near the left 4^{th} intercostal space. In some embodiments, soft-tissue retractors may be used for exposure to decrease postoperative discomfort. A subxiphoid scope may be placed directly into the pericardium and optionally the pericardium may be insufflated with gas or a liquid solution. The benefits of having a physician watching the placement of the endocardial lesions by the operating physician include the avoidance of Phrenic Nerve injury, and the confirmation of lesion location, contiguity, and transmurality.

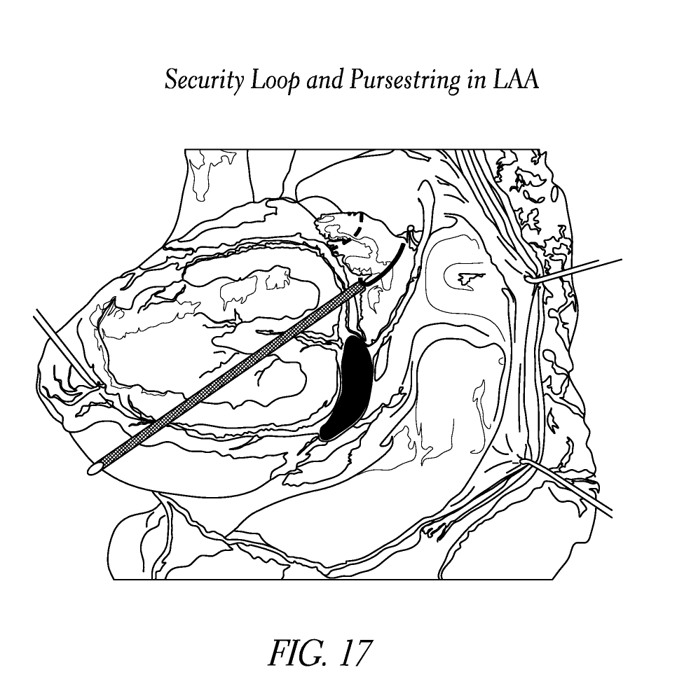

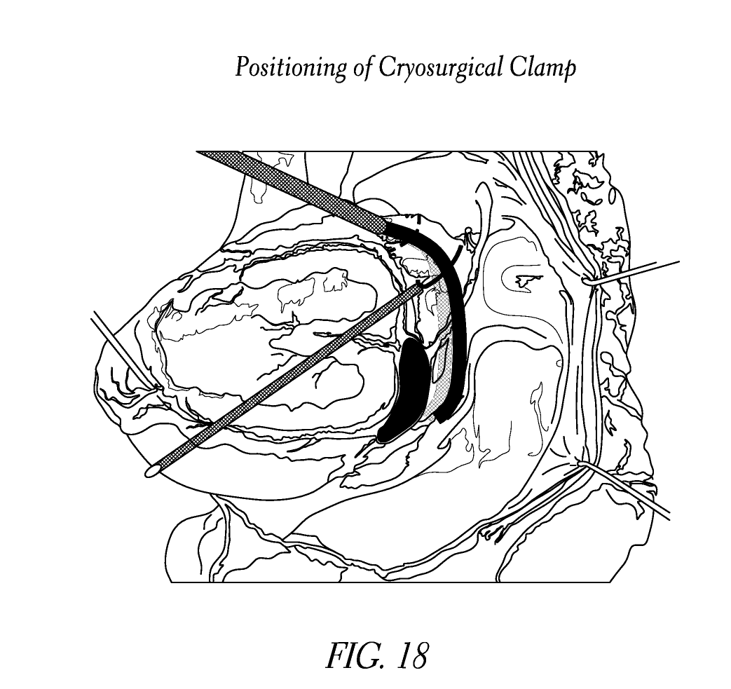

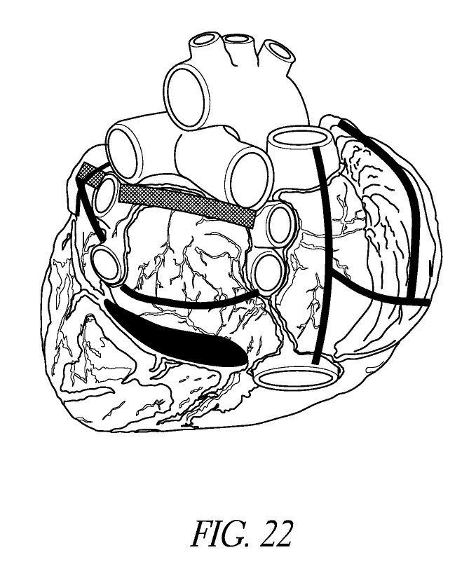

Referring now to FIGS. 16-23, in one embodiment, the first LA lesion performed may be the inferior LA lesion (FIG. 20) from the access site in the LAA and the second LA lesion may be the superior LA lesion. As shown in FIG. 17, in some embodiments a security loop may be placed around the base of the LAA using a means such as that of a Rumel Tourniquet. A purse-string suture may be placed in the tip of the LAA or lower down nearer its base.

In one embodiment, PV lesions are placed traversing the left side of the heart, with one lesion traversing a path extending across the left and right inferior pulmonary veins, and a second lesion traversing a path extending across the left and right superior pulmonary veins (the “PV lesions”). In some embodiments, the PV lesions intersect at a point in proximity to the left atrial appendage and then diverge along a superior and inferior path of traverse. The ablation energy may be of any of the forms described herein. In some embodiments, the ablation energy may be provided by a cryogen.

In some embodiments, the PV lesions may be formed using a clamp comprising an ablation member where the clamp may be passed through the left-side thorocotomy. The clamp is configured to have two opposing jaws that when actuated may open or close to apply pressure there between. One jaw of the clamp may be placed along the surface of the endocardium through a further access point through the heart. In some embodiments, access is gained through the left atrial appendage using means such as a purse string suture that may prevent the escape of blood from the beating heart. The other jaw of the clamp may remain external to the heart along the surface of the epicardium such that the wall of the heart is positioned between the jaws of the clamp and subjected to pressure when the clamp jaws are actuated closed. In one embodiment, both clap jaws comprise an ablation member configured such that a transmural lesion may be made from both the internal and external surfaces of the heart adjacent the clamp. In another embodiment, one jaw comprises an ablation member and the other jaw comprises a temperature sensor configured to detect a temperature indicative of a lesion that has reached a sufficient state of transmurality. In some embodiments, the jaws of the clamp may be a configured to allow for actuation independent of one another. Optionally, the jaws of the clamp may further comprise magnets that contribute to the clamping pressure such that lesion formation may be aided by the additional pressure.

In one embodiment for formation of the PV lesions, a probe comprising an ablation member may be used. In some embodiments, the probe may create the transmural lesion from the endocardium by being placed through an access point through the heart. In some embodiments, access is gained through the left atrial appendage using means such as a purse string suture or valved sheath that may prevent the escape of blood from the beating heart.

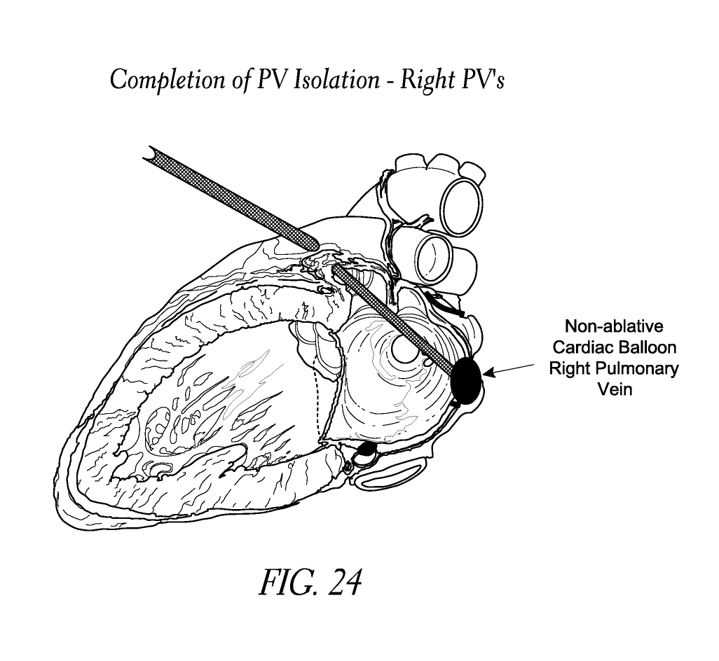

Referring now to FIGS. 24-25B, in one embodiment, the right pulmonary veins are further isolated by forming lesions that close off the divergent portion of the PV lesions (the “RPV lesions”) that may result in the PV lesion isolation pattern shown in FIG. 26. The ablation energy source may be chosen from any of those described herein. In some embodiments, a cryogen is used as the energy source.

As shown by FIG. 25A, in one embodiment for the formation of the RPV lesions, a probe comprising an ablation member at its distal end may be placed through a lumen of an endoscope placed through a subxiphoid access point. The probe is positioned on the epicardium proximate the anterior interatrial groove near the origin of the right pulmonary veins such that a lesion may be created along the perimeter of the pulmonary veins to complete the RPV lesions, thereby forming a contiguous lesion extending from a point proximate the left atrial appendage which traverses superior and inferior to the pulmonary veins and which forms a closed loop along the origins of the right pulmonary veins. FIG. 25B shows the right PV lesion isolation line across the ostia of the right pulmonary veins.

Optionally, as shown in FIGS. 24 and 25A, a balloon catheter may be positioned and inflated to expand the ostium of each of the right pulmonary veins so as to temporarily diminish the heat sink effect of cavitary blood passing through the vein in proximity to the RPV lesion as it is being formed. Secondarily, the resultant expansion of the ostium from the inflation of the balloon may expose a larger and more accessible surface area of the epicardium where the probe is placed for RPV lesion formation. Any acceptable means for catheter access may be used, with one example being the left atrial appendage using means such as a purse string suture or valved sheath that may prevent the escape of blood from the beating heart.

In one embodiment for forming the RPV lesion, a probe may be configured to comprise a shaped end that may facilitate the shaping of the RPV lesion from the endocardium. To aid in providing the desired contact pressure against the endocardium the probe distal portion may comprise a loop-like feature or plurality of loop-like features. For example, the probe distal end may be exposed from a sheath such that the loop-like feature or features are unconstrained and allowed to be formed by mechanical action or thermal action if a shape memory alloy is used. In the instance of a single loop-like feature, the feature provides locating force against the endocardium and also provides the working ablative surface for lesion formation. In the instance of a plurality of loop-like features, one or more features may be used for locating and securement while one or more features may be used for ablation. Hooks, barbs, or other such means may be further used to aid in securement in any endocardial probe embodiment.

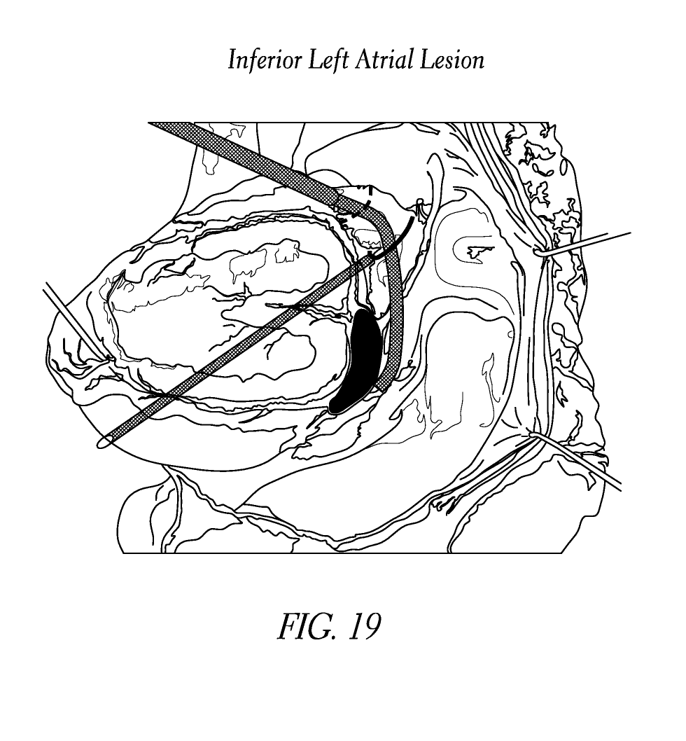

In one embodiment, the final lesion in the procedure may be the Sub-LAA lesion that may connect the end of the superior LA lesion to the end of the inferior LA lesion. Referring now to embodiments shown in FIGS. 19-25B, PV isolation may be accomplished through the LAA by placing a clamp for the superior and inferior incisions and a cryoprobe for completion of the isolation of the right PV's. The anterior sub-appendage lesion of FIGS. 26-28C may be performed to preclude atypical LA flutter.

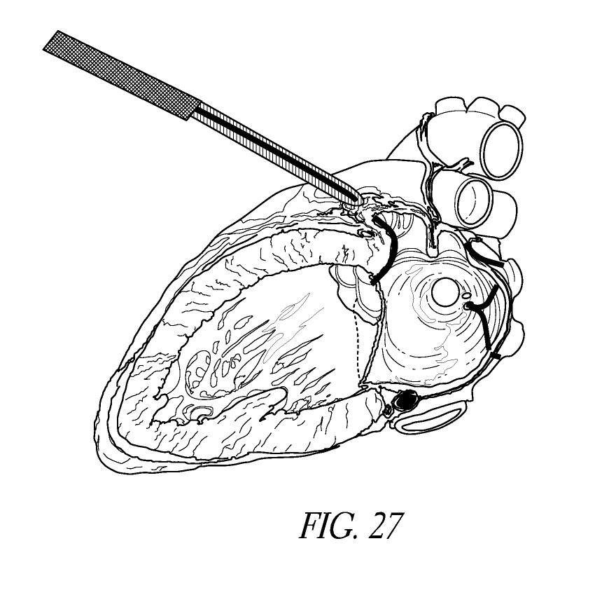

In some embodiments, the cryoprobe, which is already inside the LA via the LAA, may be pulled back while the inner probe may be curved down to reach the mitral annulus as shown in FIG. 27. Ablative energy may then be applied to create the Sub-LAA lesion and thereby complete the lesions in the LA.

As shown by FIG. 28B, the distance between the base of the LAA and the Mitral Annulus is very short and may be the only atrial myocardium ablated by the Sub-LAA lesion to stop postoperative atypical left atrial flutter. Note that the Coronary Sinus has not yet formed at this site and that the proximal circumflex coronary artery is not deep in the AV groove at this point, which again, is about 1.5 cm from its origin. In this view the LAA has been retracted upward to expose the Circumflex Coronary Artery. The fat pad in the AV groove and the coronary veins are not illustrated. The very short distance from the base of the LAA and the top of the left ventricle can be appreciated. By placing a lesion from inside the atrium, a transmural atrial lesion may be attained without thermally affecting the contents within the AV groove. Moreover, the coronary sinus has not yet formed so electrical conduction will not be able to “skirt” the atrial lesion by means of the coronary sinus as it can in the posterior left atrium.

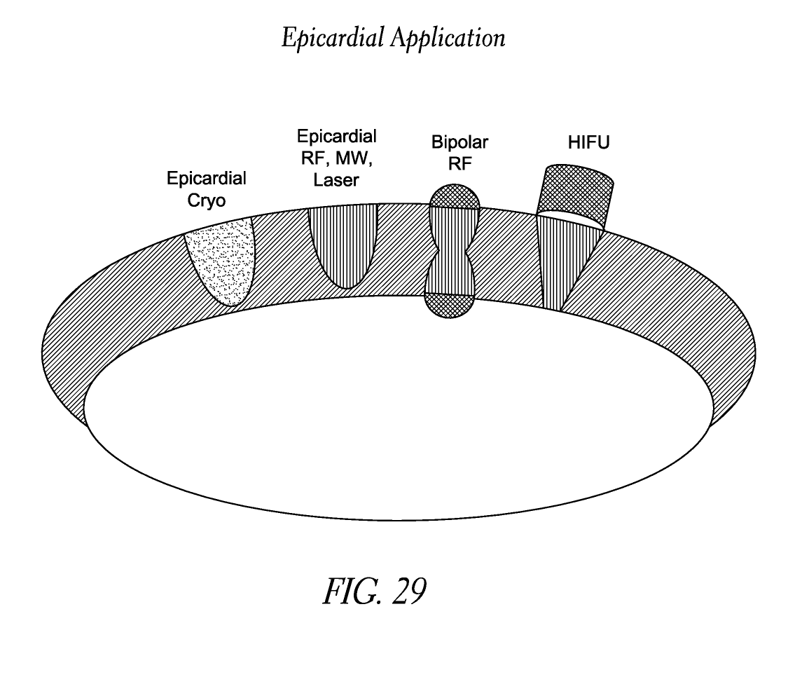

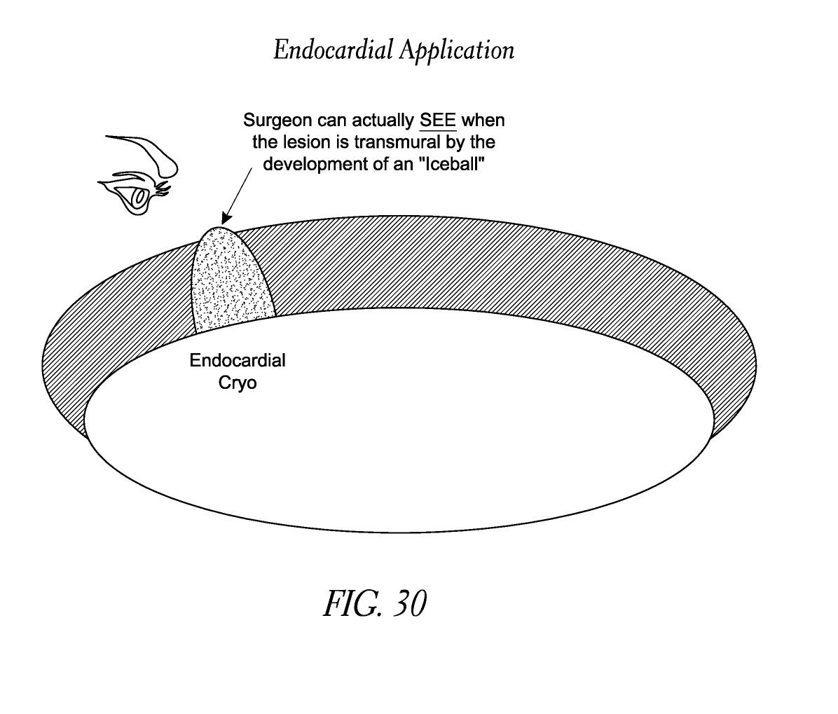

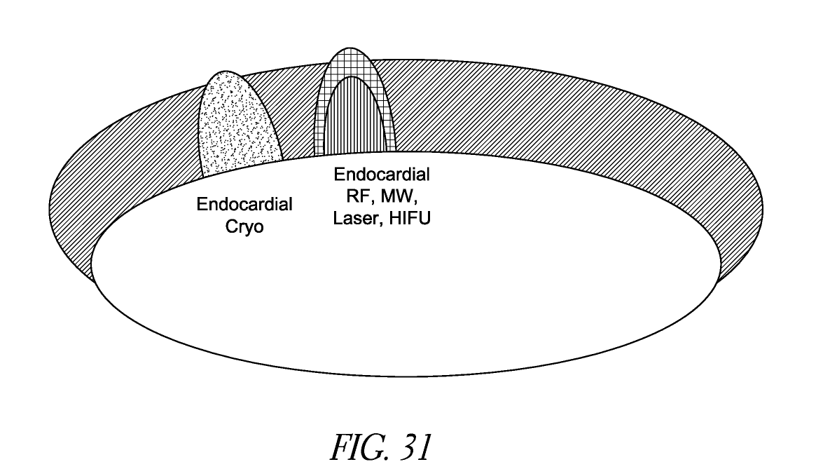

Referring now to FIGS. 29-31, although ablation energy sources may be cryogenic, microwave, laser, RF, HIFU and the like, one advantage of cryosurgery is that the operator may have direct and instant feedback by visual observation of the lesion being formed because of the “iceball” that becomes visible as shown in FIGS. 30 and 31. When performing endocardial lesions, it is advantageous to be certain of the exact location of each lesion. Further, it is advantageous to confirm that each lesion is transmural, contiguous and complete so as to avoid the persistence of macro-reentrant circuits and to avoid ablation of neighboring tissues such as nerves and the esophagus. An additional advantage of cryosurgery is when an incomplete lesion is observed, additional action may be taken to prevent failure of the procedure.

In some embodiments of the Maze-VII procedure, the steps described above comprise completion of the lesions, followed by the step of placing a surgical clip at the base of the LAA to occlude the LAA. FIG. 28D shows the lateral view of the level at which the LAA is occluded by the surgical clip.

Also described here are kits for performing any of the interventional procedures described herein. One variation of a kit may comprise a first ablation device configured to place one or more of: a lesion extending along a line between the inferior and superior vena cava, a lesion extending transversely across the right atrium and intersecting the lesion between the inferior and superior vena cava, and a lesion extending laterally along the right atrium and intersecting the transverse lesion along the right atrium; and one or more of a second ablation device configured to place a lesion in the coronary sinus; a third ablation device configured to place one or more of a lesion extending along a transverse line located below the right and left inferior pulmonary veins and a lesion extending along a transverse line located above the right and left superior pulmonary veins; a fourth ablation device configured to place a plurality of lesions extending along the anterior interatrial groove proximate the origins of the right superior and inferior pulmonary veins; and a fifth ablation device configured to place a lesion extending from the base of the left atrial appendage to a location proximate the mitral annulus, wherein the aforementioned ablation devices comprise at least one ablation member configured to deliver ablative energy to the targeted tissue, and wherein the ablative energy is selected from the group consisting of RF energy, microwave energy, cryogenic energy, laser energy, and high-frequency ultrasound energy. In some variations, the kit comprises a first and a second device as described above. In some variations, the kit comprises a first, a second and a third device as described above. In some variations, the kit comprises a first, a second, a third, and a fourth device as described above. In some variations, the kit comprises a first, a second, a third, a fourth and a fifth devices as described above. In certain variations, any of the kits described above may further comprise one or more of: a surgical clip to be placed at the base of the LAA, a surgical scope and an inflatable balloon configured to be positioned and inflated proximate the internal ostium of a pulmonary vein.

One variation of a kit may comprise a first ablation device configured to place one or more of: a lesion extending along a line between the inferior and superior vena cava, a lesion extending transversely across the right atrium and intersecting the lesion between the inferior and superior vena cava, and a lesion extending laterally along the right atrium and intersecting the transverse lesion along the right atrium, wherein the first ablation device is an ablation catheter comprising a distal portion having an ablation member configured to deliver ablative energy to the targeted tissue; and one or more of a second ablation device configured to place a lesion in the coronary sinus, wherein the second ablation device is an ablation catheter comprising an expandable structure a distal portion, wherein the expandable structure comprises an ablation member; a third ablation device configured to place one or more of a lesion extending along a transverse line located below the right and left inferior pulmonary veins and a lesion extending along a transverse line located above the right and left superior pulmonary veins, wherein the third ablation device is an ablation clamp having two opposing jaws comprising at least one ablation surface on one jaw; a fourth ablation device configured to place a plurality of lesions extending along the anterior interatrial groove proximate the origins of the right superior and inferior pulmonary veins, wherein the fourth ablation device is a flexible ablation probe comprising a flexible sheath and a flexible ablation member; and a fifth ablation device configured to place a lesion extending from the base of the left atrial appendage to a location proximate the mitral annulus wherein the fifth ablation device is a flexible ablation probe comprised of a flexible sheath and flexible ablation member; and wherein the aforementioned ablation members are configured to deliver ablative energy to the targeted tissue, wherein the ablative energy is selected from the group consisting of RF energy, microwave energy, cryogenic energy, laser energy, and high-frequency ultrasound energy. In some variations, the kit comprises a first and a second device as described above. In some variations, the kit comprises a first, a second and a third device as described above. In some variations, the kit comprises a first, a second, a third, and a fourth device as described above. In some variations, the kit comprises a first, a second, a third, a fourth and a fifth devices as described above. In certain variations, any of the kits described above may further comprise one or more of: a surgical clip to be placed at the base of the LAA, a surgical scope and an inflatable balloon configured to be positioned and inflated proximate the internal ostium of a pulmonary vein.

While several embodiments have been described in some detail, by way of example and for clarity of understanding, those of skill in the art will recognize that a variety of modification, adaptations, and changes may be employed.

1. A method of treating atrial fibrillation in a human patient comprising making in any order a series of lesions comprising:
a first lesion extending along a line between the inferior and superior vena cava;
a second lesion extending transversely across the right atrium and configured to intersect the first lesion between the inferior and superior vena cava;
a third lesion extending laterally along the right atrium and configured to intersect the second lesion;
a fourth lesion in the coronary sinus;
a fifth lesion extending along a transverse line located below the right and left inferior pulmonary veins;
a sixth lesion extending along a transverse line located above the right and left superior pulmonary veins; and
a seventh lesion comprising a plurality of lesions extending along the anterior interatrial groove proximate the origins of the right superior and inferior pulmonary veins and configured to intersect the fifth transverse lesion below the pulmonary veins and the sixth transverse lesion above the pulmonary veins, and
an eighth lesion located along a line extending from the base of the left atrial appendage to a location proximate the mitral annulus, wherein the lesions preclude the development of macro-reentrant currents.

2. The method of claim 1, further comprising placing a surgical clip at the base of the LAA to occlude the LAA.

3. The method of claim 1, wherein one or more lesions are made with an ablation device, which comprises a distal portion comprising an ablation member configured to supply ablation energy to a tissue, wherein the ablation energy is selected from the group consisting of RF energy, microwave energy, cryogenic energy, laser energy, and high-frequency ultrasound energy.

4. The method of claim 3, wherein the ablation device is an ablation catheter.

5. The method of claim 3, wherein the ablation device is an ablation clamp comprising two opposing jaws with at least one ablation member on one jaw.

6. The method of claim 5 wherein the ablation clamp comprises an ablation member on each of the two opposing jaws.

7. The method of claim 5 wherein the ablation clamp comprises an ablation member on one jaw and a temperature sensor on the opposing jaw.

8. The method of claim 1, wherein the one or more lesions along the origin of the right inferior and superior pulmonary veins are made with a flexible ablation device comprising a flexible sheath and flexible ablation member configured to supply ablation energy to a tissue, wherein the ablation energy is selected from the group consisting of RF energy, microwave energy, cryogenic energy, laser energy, and high-frequency ultrasound energy.

9. The method of claim 1, further comprising positioning an inflatable balloon proximate the internal ostium of a pulmonary vein such that inflation of the balloon increases exposure of an exterior surface of the pulmonary vein at its origin so as to improve access for an ablation device.

10. The method of claim 3, wherein the ablation device is a flexible ablation probe comprising a flexible and retractable sheath covering the ablation member, wherein the ablation member is flexible.

11. The method of claim 1, further comprising observing the making of at least one lesion through a scope passed through a subxiphoid access location.

12. The method of claim 10, wherein at least one lesion is made by passing an ablation device through an access lumen comprised within the scope.

13. The method of claim 10, further comprising insufflating the pericardium of the heart with a gas or a liquid solution, wherein insufflation aids in observation of lesion formation.

14. The method of claim 1, wherein one or more lesions on the left side of the heart are made through an access point on the left atrial appendage.

15. The method of claim 1, wherein making one or more of the fifth and sixth lesions comprises contacting an epicardial surface with one jaw of an ablation clamp and contacting an endocardial surface with another jaw of the ablation clamp such that one jaw is external to the heart and the other jaw is internal to the heart.

16. The method of claim 4 wherein the distal portion of the ablation catheter comprises an expandable ablation surface.

17. The method of claim 16, wherein the expandable ablation surface is configured to comprise one or more of a coil, a basket, a flange, and a loop-like structure.

18. The method of claim 1 further comprising observing the placement the lesion between the inferior and superior vena cava relative to the phrenic nerve through a scope.

19. The method of claim 10 wherein the ablation member of the flexible ablation probe is configured to correspond to the shape of the origins of the right pulmonary veins.

20. (canceled)

21. (canceled)

22. (canceled)

23. (canceled)

24. (canceled)

25. (canceled)

26. An ablation clamp for the treatment atrial fibrillation, the clamp comprising:
a clamp body comprising a proximal and a distal end, wherein the proximal end comprises a handle connected to an actuating structure, and wherein the distal comprises two opposing jaws operatively connected to the actuating structure, wherein the jaws are configured to be opened and closed by movement of the handle and actuating structure;
wherein the clamp comprises a curvature and is sized to allow for cardiac ablation access through an endoscope or through a thoracic incision of about 5 centimeters or less;
wherein the jaws comprise one or more ablation energy surfaces configured to come into contact when the two jaws are actuated closed, and wherein the one or more ablation energy surfaces are configured to conduct cryogenic ablation energy to a surface of the heart such that heart tissue proximate the one or more ablation energy surfaces reaches a temperature of about −30 degrees Celsius.

27. The ablation clamp of claim 25, wherein each of the two jaws comprise an ablation energy surface configured to come into contact when the two jaws are actuated closed.

28. The ablation clamp of claim 25, wherein the first of the two jaws is configured to include an ablation energy surface and wherein the second of the two jaws is configured to include a temperature sensor, wherein the ablation energy surface on the first jaw and the temperature sensor on the second jaw are configured to come into contact when the two jaws are actuated closed.

29. The ablation clamp of claim 25 wherein the source of cryogenic energy is nitrogen.

30. The ablation clamp of claim 28 wherein the nitrogen is in a supercritical state in at least a portion of the catheter.

31. The ablation clamp of claim 28 wherein the nitrogen temperature proximate the one or more energy delivery surfaces is less than about −160 degrees Celsius.

32. A flexible ablation probe for the treatment of atrial fibrillation, the probe comprising:
a probe body having a proximal and distal end with an axis there between, the proximal end comprising a handle, the distal end comprising a slideable outer sheath and an inner tip, wherein the inner tip comprises at least one ablation member for the delivery of cryogenic ablation energy, and wherein the handle is configured to control the shape and position of sheath and distal tip such that curvature of the sheath and the inner tip may be independently controlled and the slideable sheath is optionally positioned to cover or expose all or a portion of the inner tip.

33. The flexible ablation probe of claim 32 wherein the source of cryogenic energy is nitrogen.

34. The flexible ablation probe of claim 33 wherein the nitrogen is in a supercritical state in at least a portion of the probe.

35. The flexible ablation probe of claim 33 wherein the nitrogen temperature proximate to the at least one ablation energy surface is less than about −160 degrees Celsius.

36. (canceled)

37. (canceled)

38. (canceled)

39. (canceled)

40. (canceled)

41. (canceled)

42. (canceled)
